SEQUENCE LISTING (13 sequences)

SEQ ID NO: 1 - protein, 18 aa, synthetic construct
  source 1..18: note = Respiratory Syntactical Virus
ESYIGSINNITKQSACVA

SEQ ID NO: 2 - protein, 18 aa, synthetic construct
  source 1..18: note = Respiratory Syntactical Virus
ESYIGSINNITKQSASVA

SEQ ID NO: 3 - protein, 30 aa, synthetic construct
  source 1..30: note = Respiratory Syntactical Virus
NFVPCSICSNNPTCWAICKRIPNKKPGKKT

SEQ ID NO: 4 - protein, 298 aa, synthetic construct
  source 1..298: note = Respiratory Syntactical Virus
MSKNKDQRTAKTLERTWDTLNHLLFISSCLYKLNLKSVAQITLSILAMIISTSLIIAAIIFIASANHKVTPTTAIIQDATSQIKNTTPTYLTQNPQLGISPSNPSEITSQITTILASTTPGVKSTLQSTTVKTKNTTTTQTQPSKPTTKQRQNKPPSKPNNDFHFEVFNFVPCSICSNNPTCWAICKRIPNKKPGKKTTTKPTKKPTLKTTKKDPKPQTTKSKEVPTTKPTEEPTINTTKTNIITTLLTSNTTGNPELTSQMETFHSTSSEGNPSPSQVSTTSEYPSQPSSPPNTPRQ

SEQ ID NO: 5 - protein, 192 aa, synthetic construct
  source 1..192: note = Respiratory Syntactical Virus
MSRRNPCKFEIRGHCLNGKRCHFSHNYFEWPPHALLVRQNFMLNRILKSMDKSIDTLSEISGAAELDRTEEYALGVVGVLESYIGSINNITKQSACVAMSKLLTELNSDDIKKLRDNEELNSPKIRVYNTVISYIESNRKNNKQTIHLLKRLPADVLKKTIKNTLDIHKSITINNPKESTDTNDHAKNNDTT

SEQ ID NO: 6 - protein, 79 aa, synthetic construct
  source 1..79: note = Protein polypeptide; ACT-1193, ACT-2192/Pam3Cys.GM2
SKKKKNFVPCSICSNNPTCWAICKRIPNKKPGKKTSGSESYIGSINNITKQSASVASGSKKKKKKKKKKKKKKKKKKKK

SEQ ID NO: 7 - protein, 79 aa, synthetic construct
  source 1..79: note = Protein polypeptide; ACT-1216, ACT-2192/Pam3Cys.GM2
SKKKKNFVPCSICSNNPTCWAICKRIPNKKPGKKTSGSESYIGSINNITKQSACVASGSKKKKKKKKKKKKKKKKKKKK

SEQ ID NO: 8 - protein, 20 aa, synthetic construct
  source 1..20: note = Protein C-terminal poly-lysine tail
KKKKKKKKKKKKKKKKKKKK

SEQ ID NO: 9 - protein, 21 aa, synthetic construct
  source 1..21: note = Protein C-terminal poly-lysine tail
KKKKKKKKKKKKKKKKKKKKY

SEQ ID NO: 10 - protein, 74 aa, synthetic construct
  source 1..74: note = Protein ACT-1190, ACT-2182/GM2 DP/Epitope
NFVPCSICSNNPTCWAICKRIPNKKPGKKTSGSESYIGSINNITKQSASVASGSKKKKKKKKKKKKKKKKKKKK

SEQ ID NO: 11 - protein, 51 aa, synthetic construct
  source 1..51: note = Protein ACT-1213, ACT-2129/G
NFVPCSICSNNPTCWAICKRIPNKKPGKKTKKKKKKKKKKKKKKKKKKKKY

SEQ ID NO: 12 - protein, 39 aa, synthetic construct
  source 1..39: note = Protein ACT-1214, ACT-2033/M2
ESYIGSINNITKQSASVAKKKKKKKKKKKKKKKKKKKKY

SEQ ID NO: 13 - protein, 74 aa, synthetic construct; identical to SEQ ID NO: 10
  source 1..74: note = Protein ACT-1211, ACT-2182/GM2

(19) United States
(12) Patent Application Publication
(10) Pub. No.: US 2023/0414526 A1
(43) Pub. Date: Dec. 28, 2023

(54) RESPIRATORY SYNCYTIAL VIRUS ANTIGENIC COMPOSITIONS AND METHODS

(71) Applicant: Artificial Cell Technologies, Inc., New Haven, CT (US)

(72) Inventor: Thomas J. Powell, Madison, CT (US)

(21) Appl. No.: 18/188,730

(22) Filed: Mar. 23, 2023

Related U.S. Application Data
(60) Provisional application No. 63/324,824, filed on Mar. 29, 2022.

(51) Int. Cl. A61K 9/50; C07K 17/14; A61K 47/69; A61K 39/155

(52) U.S. Cl. CPC A61K 9/5052; A61K 39/155; A61K 47/6929; C07K 17/14

(57) ABSTRACT

Described herein is a composition including particles, the particles including a multilayer film, the multilayer film including two or more layers of polyelectrolytes, wherein adjacent layers include oppositely charged polyelectrolytes, wherein one of the polyelectrolytes is a designed polypeptide having the structure: (Pam3Cys or Pam2Cys)-(surface adsorption region one) -(RSV-G peptide epitope)-L1-(RSV-M2 peptide epitope)-L2-(surface adsorption region two).

### CROSS-REFERENCE TO RELATED APPLICATIONS

This application claims priority to U.S. Provisional Application 63/324,824 filed on Mar. 29, 2022, which is incorporated herein by reference in its entirety.

### SEQUENCE LISTING

The Instant Application contains a Sequence Listing which has been submitted electronically in XML format and is hereby incorporated by reference in its entirety. Said XML copy, created on Mar. 22, 2023, is named “ATE0046US2” and is 15,770 bytes in size.

### FIELD OF THE DISCLOSURE

The present disclosure relates to compositions and methods for the prevention of infection by respiratory syncytial virus, specifically multilayer film compositions containing antigenic epitopes.

### BACKGROUND

Respiratory syncytial virus (RSV) is the most important cause of serious lower respiratory tract disease in infants and young children worldwide and is also a threat to elderly and immune compromised patients. In the United States, RSV infections result in up to 126,000 infant hospitalizations and up to 60,000 elderly adult hospitalizations per year. Since natural RSV infection does not induce durable long-term immunity, patients are susceptible to re-infection with the same and different strains of virus throughout life. RSV is associated with secondary infections such as otitis media, and it may predispose young children for asthma-related illness later in life.

After more than 40 years of effort, there is no safe and effective RSV vaccine. The earliest attempts to develop a formalin-inactivated alum-precipitated RSV (FI-RSV) vaccine in the 1960's actually appeared to predispose vaccinated children to more severe disease and even death upon subsequent natural infection. The exact mechanism of this response has not been fully characterized, but it appears to be dependent on a skewing of the immune response toward an inflammatory Th2-dominant phenotype characterized by inappropriate activation of cytokine and chemokine pathways.

Given the economic impact of RSV disease, estimated at nearly $700 million per year in the US in 2004, and the life-threatening complications that can result from RSV infection in infants, elderly, and immunocompromised patients, development of safe and effective RSV vaccines is a high priority.

U.S. Pat. No. 9,487,593 describes antigenic RSV compositions and methods. There is, however, a need for improved antigenic compositions suitable for stimulating an immune response to RSV.

### SUMMARY

In an aspect a composition comprises particles, the particles comprising a multilayer film, the multilayer film comprising two or more layers of polyelectrolytes, wherein adjacent layers comprise oppositely charged polyelectrolytes, wherein one of the polyelectrolytes comprises a designed polypeptide having the structure
(Pam3Cys or Pam2Cys)-(surface adsorption region one)-(RSV-G peptide epitope) -L1-(RSV-M2peptide epitope)-L2-(surface adsorption region two)
wherein surface adsorption region one and two each independently comprise at least two amino acid residues and have the same sign of charge as the designed polypeptide, wherein the net charge per residue of the designed polypeptide is greater than or equal to 0.1,wherein the RSV-M2 peptide epitope includes (ESYIGSINNITKQSACVA) or (ESYIGSINNITKQSASVA), wherein the RSV-G peptide epitope includes (NFVPCSICSNNPTCWAICKRIPNKKPGKKT), and wherein L1 or L2 are linkers comprising 0-100 uncharged amino acid residues;
wherein the polyelectrolytes that are not the designed polypeptide comprise a polycationic material or a polyanionic material having a molecular weight of greater than 1,000 and at least 5 charges per molecule, and
wherein the multilayer film is deposited on a core particle or forms a hollow particle to provide the composition.

### BRIEF DESCRIPTION OF THE DRAWINGS

FIGS. 1A and B show the antibody response of mice immunized with ACT-1190. BALB/c mice were immunized on days 0 and 21 with the indicated doses of ACT-1190. Mice were bled one week post-boost and sera were tested in ELISA against RSV-G protein. (1A) Results show individual mice (open circles) and group means (bars) at the 1:50 dilution of sera. (1B) Results show the mean response of 16 animals per group

FIG. 2 shows the T-cell response of mice immunized with ACT-1190. On day 28, three mice/group from FIG. 1 were sacrificed and spleen cells were harvested and restimulated in vitro with RSV-M2 and RSV-G peptides in IFNγ and IL-5 ELISPOT plates. The data depict the mean±SD spots/106 cells of 3 mice/group.

FIG. 3 shows the lung RSV burden following challenge with the A2 strain. On day 35 (14 days post-boost), mice from FIG.1 were challenged with RSV A2. Five days after challenge, lungs were harvested and viral burden was measured by standard plaque assay. Results show individual mice (circles) and group means (bars); insets show number of mice completely protected from challenge (>90% reduction in viral burden), group mean percent reduction in plaque number compared to the naïve group, and P-value by Student's t-test compared to the naïve group; n=12 mice per group.

FIGS. 4A and B show the antibody response elicited by immunization with RSV microparticles. BALB/c mice were immunized with the indicated treatments on day 0 and 21. Sera collected on day 28 were tested in ELISA against RSV-G protein. (4A) Results show the mean±SEM of 10 mice per group. (4B) Sera were tested at 1:50 and plates were probed with isotype-specific detection antibodies. Results show mean±SEM of 10 mice per group.

FIG. 5 shows T-cell responses to RSV microparticles. On day 28, spleen cells were harvested from 3 mice per group from FIG. 4 and restimulated with RSV-G+M2 peptides in IFNγ and IL-5 ELISPOT plates. The data depict the mean±SD of 3 mice per group.

FIGS. 6A and B show the efficacy of RSV vaccines containing PAM3Cys. Mice from FIG. 4 were challenged with RSV on day 35 (14 days post-boost). Lungs were harvested 5 days post-challenge and viral burden was measured. (6A) Plaque assay results show individual mice (circles) and group averages (bars). (6B) qPCR results show mean±SEM of 10 mice per group. In both panels, insets show number of mice protected (>90% reduction in viral burden), average group percent reduction, and P-value (all compared to PBS group).

FIGS. 7A-C show the antibody response of mice immunized with ACT-1190, -1193, -1213, and -1214. BALB/c mice were immunized on days 0 and 21 and control mice were included as described in the text. Mice were bled 1 week post-boost and sera were tested in ELISA against RSV-G peptide. (7A) Results show the mean response of 10 animals per group. (7B) Results show individual mice (open circles) and group means (bars) at the 1:50 dilution of sera. (7C) Results show the mean±SD of 10 animals per group at the 1:50 dilution of sera.

FIGS. 8A-C show the T-cell response of mice immunized with ACT-1190, -1193, 1-1213, and -1214. On day 28, three mice/group from FIG. 7 were sacrificed and spleen cells were harvested and restimulated in vitro with RSV-M2 (8A), RSV-G (8B), or both peptides (8C) in IFNγ and IL-5 ELISPOT plates. The data depict the mean±SD spots/106 cells of 3 mice/group.

FIG. 9 shows the lung RSV burden following challenge with the A2 strain. On day 35 (14 days post-boost), mice from FIG. 7 were challenged with live RSV A2. Mice were sacrificed 5 days post-challenge, and lung viral burden was measured by standard plaque assay on Vero cells. Results show individual mice (circles) and group means (bars). Inset shows number of individual mice with complete protection, % reduction of mean viral titer per group, and P-value, for the 1214 group compared to the naïve group mean. For all other groups, 10/10 were protected, group mean was 100% protection, and P<0.0000001.

FIG. 10A-C show the number of EOS and MΦ cells in bronchoalveolar lavage (BAL) fluid. Mice from FIG. 7 were challenged with RSV on day 35, and BAL cells were collected and analyzed by flow cytometry as described in the text. The data depict the mean±SD cells/50,000 events collected of 3 mice/group at each time point.

FIGS. 11A-G show the cytokine and chemokine content in lungs following RSV challenge of immunized mice. Mice from FIG. 7 were challenged with live RSV on day 35, and BAL fluids were harvested from the lungs 6 days later. After removal of cells by centrifugation, BAL fluids were analyzed by 16-plex cytokine and chemokine ELISA. Results show mean±SD of 3 mice per group per analyte.

FIGS. 12A-E show the antibody response in BAL fluid. BAL fluids were collected from mice from FIG. 7 (3 mice per group per sacrifice day) prior to challenge (day 0) and 6 and 10 days post-challenge and tested for total IgG and against RSV-G peptide. (12A) Mean±SD peptide-specific response. (12B) Mean±SD total IgG levels. (12C), (12D), and (12E) Mean±SD isotype levels on indicated days. n=3 per group.

FIG. 13A-C show antibody response elicited by immunization with RSV-GM2 microparticles. Mice from FIG. 12 were bled on day 28 (7 days post-boost) and sera were tested in ELISA against RSV-G peptide. (13A) Results show the mean±SD of 12 mice per group. (13B) Results show individual mice (circles) and group averages (bars) at 1:50 serum dilution. (13C) Sera were tested at 1:50 and plates were probed with isotype-specific detection antibodies. Results show mean±SD of 12 mice per group.

FIG. 14 shows the lung RSV burden following challenge with the A2 strain. Mice from FIG. 12 were challenged with live RSV on day 35 (14 days post-boost) and lung virus burden was measured by standard plaque assay on Vero cells 5 days post-challenge. Results show individual mice (circles) and group means (bars); insets show % reduction of mean viral titer per group, number of individual mice with complete protection, and P-value, all compared to naïve group mean.

FIG. 15A-D shows antibody response and CCL2 levels in BAL fluid. BALB/c mice were immunized on days 0 and 21, challenged on day 35, and BAL fluid was harvested on day 41. (15A) Total IgG, mean±SD of 6 mice per group. (15B) RSV-G peptide-specific IgG in individual mice (circles) and group averages (bars). (15C) RSV-G peptide-specific IgG1 and IgG2a, mean±SD of 6 mice per group. (15D) CCL2 levels of individual mice; red circles depict values that were above the limit of detection; open circles represent values that were not above the limit of detection.

FIG. 16A-H show cytokine and chemokine content in lungs following RSV challenge of immunized mice. Mice from FIG. 14 were challenged with live RSV on day 35, and BAL fluids were harvested from the lungs 6 days later. After removal of cells by centrifugation, BAL fluids were analyzed by 16-plex cytokine and chemokine ELISA. Results show mean±SD of 3 mice per group per analyte. Red columns depict Th2 cytokines.

FIGS. 17A and B show inhibition of chemotaxis activity of RSV-G protein by antibodies induced by LbL-MP vaccination. Antibodies purified from sera from mice from FIG. 7 (FIG. 17A) and FIG. 12 (FIG. 17B) were added to lower chambers with RSV-G protein, and human PBMC were added to the upper chambers. After overnight incubation, the number of cells migrating to the lower chambers were counted, and % inhibition was calculated by comparison to control wells with only RSV-G protein in the lower chamber. The assay was performed three times in duplicate wells each time; results show mean±SD of 6 replicates per sample. 131-2G is a monoclonal antibody specific for the RSV-G CX3C epitope and was included as a positive control for inhibition of chemotaxis activity.

FIGS. 18A and B show the antibody response and weight following RSV challenge. RSV-naïve mice were immunized on days 0 and 21 (1211, 1216, FI-RSV) or infected with 106 pfu of live RSV on day 0. (18A) Mice were bled on day 28 and antibody levels were measured by ELISA on RSV-G-coated plates. Results show individual mice (open circles) and the mean±SD of 10 mice per group (red bars) at 1:50 serum dilution. (18B) Mice were challenged on day 35 (day 0 post-challenge on the x-axis) and weighed every day starting the day of challenge (day 0). Results show the average percent of starting weight for 10 mice/group through day 5 when 7/group were sacrificed for plaque assay, and the remaining 3 mice/group through day 8 when they were sacrificed for BAL fluid and cells.

FIGS. 19A and B show the viral burden in lungs of BALB/c mice immunized with RSV microparticles or FI-RSV or convalescent from RSV infection. Mice from FIG. 18 were challenged with 106 pfu 2 weeks after boost, sacrificed 5 days later and lung viral burden was measured. [N]=naïve; [+]=challenged; [−]=not challenged. (19A) Plaque assay results. (19B) qPCR results. Both panels show individual mice (red =group average). Insets show group average % reduction vs naïve group average, number of animals with >90% reduction in viral burden, and P-values vs naïve challenged group by Student's t-test where [NS]=not significant and [*]<0.005.

FIGS. 20A and B show fluorescence staining of lung cells collected via lavage 8 days after viral challenge. Mice from FIG. 18 were sacrificed 8 days post-challenge and BAL cells were harvested. Cells were stained with a cocktail of antibodies against CD3/CD4/CD8 (20A) or against CD45/CD11c/SiglecF (20B) and analyzed as described in the text. Results are shown as fold increase relative to the naïve group average for 3 individual mice per group.

FIG. 21 shows scatter plots of lung cells collected via lavage 8 days after viral challenge of naïve (left) and FI-RSV-immunized (right) mice from FIG. 18. The triangular region, R1, contains the T-cells while the polygonal region, R4, contains the larger and more granular CD45+ eosinophils.

FIGS. 22A and B show Th1 (22A) and Th2 (22B) cytokine levels in lung homogenates and BAL fluid of mice from FIG. 18. Lungs were harvested, homogenized, and clarified 5 days after viral challenge (7/group). Cytokines were measured using a fluorescent bead-based multi-analyte flow assay kit. Data are presented as the average fold change for each group vs. naïve controls. The groups designated ‘naïve, nc’ were naïve animals that were anesthetized but not challenged

The above-described and other features will be appreciated and understood by those skilled in the art from the following detailed description, drawings, and appended claims.

### DETAILED DESCRIPTION

Disclosed herein are multilayer films and particles comprising polypeptide epitopes from RSV, wherein the multilayer films are capable of eliciting an immune response in a host upon administration to the host. Specifically, described herein are specific designed TLR-targeted peptides which provide multilayer films that advantageously favor a more balanced immune response (much lower levels of Th2 cytokines) compared to the same peptides with no TLR ligand. The immune responses of the constructs described herein are comparable to mice convalescent from a previous infection with low dose live RSV prior to challenge. The TLR-targeted peptide and multilayer films are advantageous because it is widely believed that the problem with the formalin-inactivated vaccine trials of the 1960s, in which children became sicker or even died after natural exposure compared to unvaccinated children, was caused by Th2 inflammatory responses due to the formalin inactivation disrupting the virus:TLR interaction during vaccination. The immunogenic multilayer film constructs described herein advantageously avoid these problems of prior RSV vaccines.

In an aspect, a composition comprises particles, the particles comprising a multilayer film, the multilayer film comprising two or more layers of polyelectrolytes, wherein adjacent layers comprise oppositely charged polyelectrolytes, wherein one of the polyelectrolytes comprises a designed polypeptide having the structure
(Pam3Cys or Pam2Cys)-(surface adsorption region one)-(RSV-G peptide epitope) -L1-(RSV-M2peptide epitope)-L2-(surface adsorption region two)
wherein surface adsorption region one and two each independently comprise at least two amino acid residues and have the same sign of charge as the designed polypeptide, wherein the net charge per residue of the designed polypeptide is greater than or equal to 0.1, wherein the RSV-M2 peptide epitope includes (ESYIGSINNITKQSACVA; SEQ ID NO: 1) or (ESYIGSINNITKQSASVA; SEQ ID NO: 2), wherein the RSV-G peptide epitope includes (NFVPCSICSNNPTCWAICKRIPNKKPGKKT; SEQ ID NO: 3), wherein L1 or L2 are linkers comprising 0-100 uncharged amino acid residues; wherein the polyelectrolytes that are not the designed polypeptide comprise a polycationic material or a polyanionic material having a molecular weight of greater than 1,000 and at least 5 charges per molecule, and wherein the multilayer film is deposited on a core particle or forms a hollow particle to provide the composition.

As described herein a multilayer film comprises alternating layers of oppositely charged polyelectrolytes in which one layer, such as the outermost layer, comprises a designed polypeptide represented by: (Pam3Cys or Pam2Cys)-(surface adsorption region one)-(RSV-G peptide epitope)-L1-(RSV-M2 peptide epitope)-L2-(surface adsorption region two). The polyelectrolytes in the multilayer film that are not the designed polypeptide comprise a polycationic material or a polyanionic material having a molecular weight of greater than 1,000 and at least 5 charges per molecule, and wherein the multilayer film is deposited on a core particle or forms a hollow particle to provide the composition.

More specifically, polyelectrolyte multilayer films are thin films (e.g., a few nanometers to micrometers thick) composed of alternating layers of oppositely charged polyelectrolytes. Such films can be formed by layer-by-layer assembly on a substrate. In electrostatic layer-by-layer self-assembly (“ELBL”), the physical basis of association of polyelectrolytes is electrostatic attraction. Film buildup is possible because the sign of the surface charge density of the film reverses on deposition of successive layers. The generality and relative simplicity of the ELBL film process permits the deposition of many different types of polyelectrolyte onto many different types of surface. Polypeptide multilayer films are a subset of polyelectrolyte multilayer films, comprising at least one layer comprising a charged polypeptide, herein referred to as a designed polypeptide. A key advantage of polypeptide multilayer films over films made from other polymers is their biocompatibility. ELBL films can also be used for encapsulation. Applications of polypeptide films and microcapsules include, for example, nano-reactors, biosensors, artificial cells, and drug delivery vehicles.

The term “polyelectrolyte” includes polycationic and polyanionic materials having a molecular weight of greater than 1,000 and at least 5 charges per molecule. Suitable polycationic materials include, for example, polypeptides and polyamines. Polyamines include, for example, a polypeptide such as poly-L-lysine (PLL) or poly-L-ornithine, polyvinyl amine, poly(aminostyrene), poly(aminoacrylate), poly (N-methyl aminoacrylate), poly (N-ethylaminoacrylate), poly(N,N-dimethyl aminoacrylate), poly(N,N-diethylaminoacrylate), poly(aminomethacrylate), poly(N-methyl amino- methacrylate), poly(N-ethyl aminomethacrylate), poly(N,N-dimethyl aminomethacrylate), poly(N,N-diethyl aminomethacrylate), poly(ethyleneimine), poly (diallyl dimethylammonium chloride), poly(N,N,N-trimethylaminoacrylate chloride), poly(methyacrylamidopropyltrimethyl ammonium chloride), chitosan and combinations comprising one or more of the foregoing polycationic materials. Suitable polyanionic materials include, for example, a polypeptide such as poly-L-glutamic acid (PGA) and poly-L-aspartic acid, a nucleic acid such as DNA and RNA, alginate, carrageenan, furcellaran, pectin, xanthan, hyaluronic acid, heparin, heparan sulfate, chondroitin sulfate, dermatan sulfate, dextran sulfate, poly(meth)acrylic acid, oxidized cellulose, carboxymethyl cellulose, acidic polysaccharides, and croscarmelose, synthetic polymers and copolymers containing pendant carboxyl groups, and combinations comprising one or more of the foregoing polyanionic materials. In one embodiment, the RSV epitope and the polyelectrolyte have the same sign of charge.

In an aspect, a stable multilayer film is a film that once formed, retains more than half its components after incubation in PBS at 37° C. for 24 hours.

A designed polypeptide means a polypeptide that has sufficient charge for stable binding to an oppositely charged surface, that is, a polypeptide that can be deposited into a layer of a multilayer film wherein the driving force for film formation is electrostatics. In another embodiment, the solubility of the designed polypeptide at pH 4 to 10 is greater than or equal to about 0.1 mg/mL. In another embodiment, the solubility of the designed polypeptide at pH 4 to 10 is greater than or equal to about 1 mg/mL. The solubility is a practical limitation to facilitate deposition of the polypeptides from aqueous solution. A practical upper limit on the degree of polymerization of an antigenic polypeptide is about 1,000 residues. It is conceivable, however, that longer composite polypeptides could be realized by an appropriate method of synthesis.

In specific embodiments, the magnitude of the net charge per residue of the designed polypeptide is greater than or equal to 0.1, 0.2, 0.3, 0.4 or 0.5 at pH 7.0. In one embodiment, the ratio of the number of charged residues of the same polarity minus the number of residues of the opposite polarity to the total number of residues in the polypeptide is greater than or equal to 0.5 at pH 7.0. In other words, the magnitude of the net charge per residue of the polypeptide is greater than or equal to 0.5. While there is no absolute upper limit on the length of the polypeptide, in general, designed polypeptides suitable for ELBL deposition have a practical upper length limit of 1,000 residues. Designed polypeptides can include sequences found in nature such as RSV epitopes as well as regions that provide functionality to the peptides such as charged regions also referred to herein as surface adsorption regions, which allow the designed polypeptides to be deposited into a polypeptide multilayer film.

Positively-charged (basic) naturally-occurring amino acids at pH 7.0 are arginine (Arg), histidine (His), ornithine (Orn), and lysine (Lys). Negatively-charged (acidic) naturally-occurring amino acid residues at pH 7.0 are glutamic acid (Glu) and aspartic acid (Asp). A mixture of amino acid residues of opposite charge can be employed so long as the overall net ratio of charge meets the specified criteria. In one embodiment, a designed polypeptide is not a homopolymer. In another embodiment, a designed polypeptide is unbranched.

The designed polypeptide comprises a Pam3Cys or Pam3Cys at the N-terminus. Pam3Cys and Pam2Cys are TLR ligands. Pam3Cys ([N-palmitoyl-S-[2,3-bis(palmitoyloxy)propyl]cysteine]) is a triacyl bacterial lipoprotein TLR1 ligand. Pam2Cys (Pam2Cys [S-[2,3-bis(palmitoyloxy)propyl]cysteine]) is a diacyl bacterial lipoprotein TLR2 ligand. While the experiments described herein use Pam3Cys, similar results are expected with Pam2Cys.

Pam3Cys and Pam2Cys can be covalently coupled to a polypeptide chain by standard polypeptide synthesis chemistry. For example, Pam3Cys may be covalently linked to an antigenic polypeptide through direct covalent linkage via an amide bond formed between the carboxylic acid of Pam3Cys-OH (commercially available from Bachem, Inc.) to the N-terminal of a peptide. A convenient way to accomplish this reaction is to couple Pam3Cys-OH in the presence of an amide bond forming reagent such as HBTU (O-Benzotriazole-N,N,N′,N′-tetramethyl-uronium-hexafluoro-phosphate), HATU (2-(1H-7-Azabenzotriazol-1-yl)-1,1,3,3-tetramethyl uronium hexafluorophosphate Methanaminium), or DIPCDI (N,N′-Diisopropylcarbodiimide) to a synthetic peptide on a solid phase synthesis resin bead. The progress of the coupling reaction can be monitored colorimetrically by ninhydrin assay and, following completion, excess Pam3Cys-OH and other reagents can be washed away. The synthetic Pam3Cys peptide conjugate is cleaved from the resin and purified by chromatography. For example, Pam3Cys peptides can be purified by reverse phase HPLC using a C4 column and a water/isopropanol gradient. An advantage of this approach is that the Pam3Cys/antigenic polypeptide is strictly controlled in a 1:1 ratio.

In another embodiment, Pam3Cys-OH is conjugated specifically to the side chain ε-amine of lysine residue, either specifically to a resin bound peptide as described above, or nonspecifically to an unprotected peptide or protein using water soluble coupling reagent such as EDC/sulfo-NHS. The product of that reaction is purified, for example, by gel permeation chromatography or dialysis, then incorporated into a particle by LBL or other methods.

The designed peptide comprises two RSV epitopes, an RSV-M2 epitope and an RSV-G epitope. The RSV-M2 peptide epitope includes (ESYIGSINNITKQSACVA; SEQ ID NO: 1) or (ESYIGSINNITKQSASVA; SEQ ID NO: 2), wherein the RSV-G peptide epitope includes (NFVPCSICSNNPTCWAICKRIPNKKPGKKT; SEQ ID NO: 3). The full sequences of RSV-M2 and RSV-G are provided below.

| RSV-G- | | |
|---|---|---|
| SEQ ID NO: 4 | | |
| 1 | MSKNKDQRTA KTLERTWDTL NHLLFISSCL YKLNLKSVAQ ITLSILAMII STSLIIAAII | 60 |
| 61 | FIASANHKVT PTTAIIQDAT SQIKNTTPTY LTQNPQLGIS PSNPSEITSQ ITTILASTTP | 120 |
| 121 | GVKSTLQSTT VKTKNTTTTQ TQPSKPTTKQ RQNKPPSKPN NDFHFEVENF VPCSICSNNP | 180 |
| 181 | TCWAICKRIP NKKPGKKTTT KPTKKPTLKT TKKDPKPQTT KSKEVPTTKP TEEPTINTTK | 240 |
| 241 | TNIITTLLTS NTTGNPELTS QMETFHSTSS EGNPSPSQVS TTSEYPSQPS SPPNTPRQ | 298 |
| | RSV-M2- | |
| SEQ ID NO: 5 | | |
| 1 | MSRRNPCKFE IRGHCLNGKR CHESHNYFEW PPHALLVRQN FMLNRILKSM DKSIDTLSEI | 1 |
| 61 | SGAAELDRTE EYALGVVGVL ESYIGSINNI TKQSACVAMS KLLTELNSDD IKKLRDNEEL | 60 |
| 121 | NSPKIRVYNT VISYIESNRK NNKQTIHLLK RLPADVLKKT IKNTLDIHKS ITINNPKEST | 180 |

The designed peptide includes two surface adsorption regions which provide sufficient charge for the peptide to assemble into a multilayer film. In an aspect, each of the first and second surface adsorption region independently comprises at least 2, 3, 4, 5, 6, 7, or 8 amino acid residues and has the same sign of charge as the designed polypeptide. The first and second surface adsorption region may be the same or different.

As used herein, a surface adsorption region is a charged region of a designed polypeptide that advantageously provides sufficient charge so that a peptide containing an epitope from RSV, for example, can be deposited into a multilayer film.

The designed polypeptide also includes amino acid linkers L1 and L2 which comprise 0-100 uncharged amino acid residues. In an aspect, L1 and L2 are SGS

Specific designed polypeptides include:

SEQ ID NO: 6
Pam3CSKKKKNFVPCSICSNNPTCWAICKRIPNKKPGKKTSGSESYIGSI
NNITKQSASVASGSKKKKKKKKKKKKKKKKKKKK;
SEQ ID NO: 7
Pam3CSKKKKNFVPCSICSNNPTCWAICKRIPNKKPGKKTSGSESYIGSI
NNITKQSACVASGSKKKKKKKKKKKKKKKKKKKK;

In one embodiment, the multilayer film is deposited on a core particle, such as a CaCO3 nanoparticle, a latex particle, or an iron particle. Particle sizes on the order of 5 nanometers (nm) to 50 micrometers (μm) in diameter are particularly useful. Particles made of other materials can also be used as cores provided that they are biocompatible, have controllable size distribution, and have sufficient surface charge (either positive or negative) to bind polyelectrolyte peptides. Examples include nanoparticles and microparticles made of materials such as polylactic acid (PLA), polylactic acid glycolic acid copolymer (PLGA), polyethylene glycol (PEG), chitosan, hyaluronic acid, gelatin, or combinations thereof. Core particles could also be made of materials that are believed to be inappropriate for human use provided that they can be dissolved and separated from the multilayer film following film fabrication. Examples of the template core substances include organic polymers such as latex or inorganic materials such as silica.

One design concern is control of the stability of polypeptide ELBL films. Ionic bonds, hydrogen bonds, van der Waals interactions, and hydrophobic interactions contribute to the stability of multilayer films. In addition, covalent disulfide bonds formed between sulfhydryl-containing amino acids in the polypeptides within the same layer or in adjacent layers can increase structural strength. Sulfhydryl-containing amino acids include cysteine and homocysteine, and these residues can be readily incorporated into synthetic designed peptides. In addition sulfhydryl groups can be incorporated into polyelectrolyte homopolymers such as poly-L-lysine or poly-L-glutamic acid by methods well described in the literature. Sulfhydryl-containing amino acids can be used to “lock” (bond together) and “unlock” layers of a multilayer polypeptide film by a change in oxidation potential. Also, the incorporation of a sulfhydryl-containing amino acid in a designed polypeptide enables the use of relatively short peptides in thin film fabrication, by virtue of intermolecular disulfide bond formation.

In one embodiment, the designed sulfhydryl-containing polypeptides, whether synthesized chemically or produced in a host organism, are assembled by ELBL in the presence of a reducing agent to prevent premature disulfide bond formation. Following film assembly, the reducing agent is removed and an oxidizing agent is added. In the presence of the oxidizing agent disulfide bonds form between sulfhydryl groups, thereby “locking” together the polypeptides within layers and between layers where thiol groups are present. Suitable reducing agents include dithiothreitol (DTT), 2-mercaptoethanol (BME), reduced glutathione, tris(2-carboxyethyl)phosphine hydrochloride (TCEP), and combinations of more than one of these chemicals. Suitable oxidizing agents include oxidized glutathione, tert-butylhydroperoxide (t-BHP), thimerosal, diamide, 5,5′-dithio-bis-(2-nitro-benzoic acid) (DTNB), 4,4′-dithiodipyridine, sodium bromate, hydrogen peroxide, sodium tetrathionate, porphyrindin, sodium orthoiodosobenzoate, and combinations of more than one of these chemicals.

As an alternative to disulfide bonds, chemistries that produce other covalent bonds can be used to stabilize ELBL films. For films comprised of polypeptides, chemistries that produce amide bonds are particularly useful. In the presence of appropriate coupling reagents, acidic amino acids (those with side chains containing carboxylic acid groups such as aspartic acid and glutamic acid) will react with amino acids whose side chains contain amine groups (such as lysine and ornithine) to form amide bonds. Amide bonds are more stable than disulfide bonds under biological conditions and amide bonds will not undergo exchange reactions. Many reagents can be used to activate polypeptide side chains for amide bonding. Carbodiimide reagents, such as the water soluble 1-ethyl-3-(3-dimethylaminopropyl) carbodiimide (EDC) will react with aspartic acid or glutamic acid at slightly acidic pH, forming an intermediate product that will react irreversibly with an amine to produce an amide bond. Additives such as N-hydroxysuccinimide are often added to the reaction to accelerate the rate and efficiency of amide formation. After the reaction the soluble reagents are removed from the nanoparticles or microparticles by centrifugation and aspiration. Examples of other coupling reagents include diisopropylcarbodiimide, HBTU, HATU, HCTU, TBTU, and PyBOP. Examples of other additives include sulfo-N-hydroxysuccinimide, 1-hydroxbenzotriazole, and 1-hydroxy-7-aza-benzotriazole. The extent of amide cross linking can be controlled by modulating the stoichiometry of the coupling reagents, the time of reaction, or the temperature of the reaction, and can be monitored by techniques such as Fourier transform—infrared spectroscopy (FT-IR).

Covalently cross-linked ELBL films have desirable properties such as increased stability. Greater stability allows for more stringent conditions to be used during nanoparticle, microparticle, nanocapsule, or microcapsule fabrication. Examples of stringent conditions include high temperatures, low temperatures, cryogenic temperatures, high centrifugation speeds, high salt buffers, high pH buffers, low pH buffers, filtration, and long term storage.

A method of making a polyelectrolyte multilayer film comprises depositing a plurality of layers of oppositely charged chemical species on a substrate. At least one layer, preferably the outermost layer, comprises a designed polypeptide as described herein. Successively deposited polyelectrolytes will have opposite net charges. In one embodiment, deposition of a polyelectrolyte comprises exposing the substrate to an aqueous solution comprising a polyelectrolyte at a pH at which it has a suitable net charge for ELBL. In other embodiments, the deposition of a polyelectrolyte on the substrate is achieved by sequential spraying of solutions of oppositely charged polypeptides. In yet other embodiments, deposition on the substrate is by simultaneous spraying of solutions of oppositely charged polyelectrolytes.

In the ELBL method of forming a multilayer film, the opposing charges of the adjacent layers provide the driving force for assembly. It is not critical that polyelectrolytes in opposing layers have the same net linear charge density, only that opposing layers have opposite charges. One standard film assembly procedure by deposition includes forming aqueous solutions of the polyions at a pH at which they are ionized (i.e., pH 4-10), providing a substrate bearing a surface charge, and alternating immersion of the substrate into the charged polyelectrolyte solutions. The substrate is optionally washed in between deposition of alternating layer.

The concentration of polyelectrolyte suitable for deposition of the polyelectrolyte can readily be determined by one of ordinary skill in the art. An exemplary concentration is 0.1 to 10 mg/mL. For typical non-polypeptide polyelectrolytes such as poly(acrylic acid) and poly(allylamine hydrochloride), typical layer thicknesses are about 3 to about 5 Å, depending on the ionic strength of solution. Short polyelectrolytes typically form thinner layers than long polyelectrolytes. Regarding film thickness, polyelectrolyte film thickness depends on humidity as well as the number of layers and composition of the film. For example, PLL/PGA films 50 nm thick shrink to 1.6 nm upon drying with nitrogen. In general, films of 1 nm to 100 nm or more in thickness can be formed depending on the hydration state of the film and the molecular weight of the polyelectrolytes employed in the assembly.

In addition, the number of layers required to form a stable polyelectrolyte multilayer film will depend on the polyelectrolytes in the film. For films comprising only low molecular weight polypeptide layers, a film will typically have 4 or more bilayers of oppositely charged polypeptides. For films comprising high molecular weight polyelectrolytes such as poly(acrylic acid) and poly(allylamine hydrochloride), films comprising a single bilayer of oppositely charged polyelectrolyte can be stable. Studies have shown that polyelectrolyte films are dynamic. The polyelectrolytes contained within a film can migrate between layers and can exchange with soluble polyelectrolytes of like charge when suspended in a polyelectrolyte solution. Moreover, polyelectrolyte films can disassemble or dissolve in response to a change in environment such as temperature, pH, ionic strength, or oxidation potential of the suspension buffer. Thus, some polyelectrolytes and particularly peptide polyelectrolytes exhibit transient stability. The stability of peptide polyelectrolyte films can be monitored by suspending the films in a suitable buffer under controlled conditions for a fixed period of time, and then measuring the amounts of the peptides within the film with a suitable assay such as amino acid analysis, HPLC assay, or fluorescence assay. Peptide polyelectrolyte films are most stable under conditions that are relevant to their storage and usage as vaccines, for example in neutral buffers and at ambient temperatures such as 4° C. to 37° C. Under these conditions stable peptide polyelectrolyte films will retain most of their component peptides for at least 24 hours and often up to 14 days and beyond.

For synthesis of the designed polypeptides, each of the independent regions (e.g., RSV epitopes and surface adsorption regions) of the designed polypeptide can be synthesized separately by solution phase peptide synthesis, solid phase peptide synthesis, or genetic engineering of a suitable host organism. Solution phase peptide synthesis is the method used for production of most of the approved peptide pharmaceuticals on the market today. A combination of solution phase and solid phase methods can be used to synthesize relatively long peptides and even small proteins. Peptide synthesis companies have the expertise and experience to synthesize difficult peptides on a fee-for-service basis. The syntheses are performed under good manufacturing practices (GMP) conditions and at a scale suitable for clinical trials and commercial drug launch.

Alternatively, the various independent regions can be synthesized together as a single polypeptide chain by solution-phase peptide synthesis, solid phase peptide synthesis or genetic engineering of a suitable host organism. The choice of approach in any particular case will be a matter of convenience or economics.

If the various RSV epitopes and surface adsorption regions are synthesized separately, once purified, for example, by ion exchange chromatography or by high performance liquid chromatography, they are joined by peptide bond synthesis. That is, the N-terminus of the surface adsorption region and the C-terminus of the RSV epitope are covalently joined to produce the designed polypeptide. Alternatively, the C-terminus of the surface adsorption region and the N-terminus of the RSV epitope are covalently joined to produce the designed polypeptide. The individual fragments can be synthesized by solid phase methods and obtained as fully protected, fully unprotected, or partially protected segments. The segments can be covalently joined in a solution phase reaction or solid phase reaction. If one polypeptide fragment contains a cysteine as its N-terminal residue and the other polypeptide fragment contains a thioester or a thioester precursor at its C-terminal residue the two fragments will couple spontaneously in solution by a specific reaction commonly known (to those skilled in the art) as Native Ligation. Native Ligation is a particularly attractive option for designed peptide synthesis because it can be performed with fully deprotected or partially protected peptide fragments in aqueous solution and at dilute concentrations.

In one embodiment, the RSV epitopes and/or surface adsorption regions are joined by peptidic or non-peptidic linkages as described in U.S. Pat. No. 7,723,294, incorporated herein by reference for its teaching of the use of non-peptidic linkages to join segments of polypeptides for use in multilayer films. Suitable non-peptidic linkers include, for example, alkyl linkers such as —NH—(CH2)s-C(O)—, wherein s=2-20. Alkyl linkers are optionally substituted by a non-sterically hindering group such as lower alkyl (e.g., C1-C6), lower acyl, halogen (e.g., Cl, Br), CN, NH2, phenyl, and the like. Another exemplary non-peptidic linker is a polyethylene glycol linker such as —NH—(CH2-CH2-O)n, —C(O)— wherein n is such that the linker has a molecular weight of 100 to 5000 Da, specifically 100 to 500 Da. Many of the linkers described herein are available from commercial vendors in a form suitable for use in solid phase peptide synthesis.

Further disclosed herein is an immunogenic composition, said immunogenic composition comprising a multilayer film comprising two or more layers of polyelectrolytes, wherein adjacent layers comprise oppositely charged polyelectrolytes, wherein one layer comprises an RSV epitope. The immunogenic composition optionally further comprises one or more layers comprising a designed polypeptide.

Also included are methods if administering the compositions described herein to an individual in need of immunization from RSV.

In an aspect, the particle described herein elicits an IFNγ T-cell phenotype and elicits fewer IL-5-producing T-cells compared to a designed peptide lacking the (Pam3Cys or Pam2Cys). In another aspect, the composition elicits a broader isotype distribution in the RSV-G protein-specific antibody response compared to a designed peptide lacking the (Pam3Cys or Pam2Cys). In a further aspect, the composition elicits IgG1, IgG2a and IgG2b isotypes.

The immunogenicity of an immunogenic composition may be enhanced in a number of ways. In one embodiment, the multilayer film optionally comprises one or more additional immunogenic bioactive molecules. Although not necessary, the one or more additional immunogenic bioactive molecules will typically comprise one or more additional antigenic determinants. Suitable additional immunogenic bioactive molecules include, for example, a drug, a protein, an oligonucleotide, a nucleic acid, a lipid, a phospholipid, a carbohydrate, a polysaccharide, a lipopolysaccharide, a low molecular weight immune stimulatory molecule, or a combination comprising one or more of the foregoing bioactive molecules. Other types of additional immune enhancers include a functional membrane fragment, a membrane structure, a virus, a pathogen, a cell, an aggregate of cells, an organelle, or a combination comprising one or more of the foregoing bioactive structures.

In one embodiment, the multilayer film optionally comprises one or more additional bioactive molecules. The one or more additional bioactive molecule can be a drug. Alternatively, the immunogenic composition is in the form of a hollow shell or a coating surrounding a core. The core comprises a variety of different encapsulants, for example, one or more additional bioactive molecules, including, for example, a drug. Thus, the immunogenic compositions designed as described herein could also be used for combined therapy, e.g., eliciting an immune response and for targeted drug delivery. Micron-sized “cores” of a suitable therapeutic material in “crystalline” form can be encapsulated by immunogenic composition comprising the antigenic polypeptides, and the resulting microcapsules could be used for drug delivery. The core may be insoluble under some conditions, for instance high pH or low temperature, and soluble under the conditions where controlled release will occur. The surface charge on the crystals can be determined by ζ-potential measurements (used to determine the charge in electrostatic units on colloidal particles in a liquid medium). The rate at which microcapsule contents are released from the interior of the microcapsule to the surrounding environment will depend on a number of factors, including the thickness of the encapsulating shell, the antigenic polypeptides used in the shell, the presence of disulfide bonds, the extent of cross-linking of peptides, temperature, ionic strength, and the method used to assemble the peptides. Generally, the thicker the capsule, the longer the release time.

In another embodiment, the additional immunogenic biomolecule is a nucleic acid sequence capable of directing host organism synthesis of a desired immunogen or interfering with the expression of genetic information from a pathogen. In the former case, such a nucleic acid sequence is, for example, inserted into a suitable expression vector by methods known to those skilled in the art. Expression vectors suitable for producing high efficiency gene transfer in vivo include retroviral, adenoviral and vaccinia viral vectors. Operational elements of such expression vectors include at least one promoter, at least one operator, at least one leader sequence, at least one terminator codon, and any other DNA sequences necessary or preferred for appropriate transcription and subsequent translation of the vector nucleic acid. In particular, it is contemplated that such vectors will contain at least one origin of replication recognized by the host organism along with at least one selectable marker and at least one promoter sequence capable of initiating transcription of the nucleic acid sequence. In the latter case, multiple copies of such a nucleic acid sequence will be prepared for delivery, for example, by encapsulation of the nucleic acids within a polypeptide multilayer film in the form of a capsule for intravenous delivery.

In construction of a recombinant expression vector, it should additionally be noted that multiple copies of the nucleic acid sequence of interest and its attendant operational elements may be inserted into each vector. In such an embodiment, the host organism would produce greater amounts per vector of the desired protein. The number of multiple copies of the nucleic acid sequence which may be inserted into the vector is limited only by the ability of the resultant vector due to its size, to be transferred into and replicated and transcribed in an appropriate host microorganism.

In one embodiment, the multilayer film/immunogenic composition evokes a response from the immune system to a pathogen. In one embodiment, a vaccine composition comprises an immunogenic composition in combination with a pharmaceutically acceptable carrier. Thus, a method of vaccination against a pathogenic disease comprises the administering to a subject in need of vaccination an effective amount of the immunogenic composition.

Pharmaceutically acceptable carriers include, but are not limited to, large, slowly metabolized macromolecules such as proteins, polysaccharides, polylactic acids, polyglycolic acids, polymeric amino acids, amino acid copolymers, inactive virus particles, and the like. Pharmaceutically acceptable salts can also be used in the composition, for example, mineral salts such as hydrochlorides, hydrobromides, phosphates, or sulfates, as well as the salts of organic acids such as acetates, proprionates, malonates, or benzoates. The composition can also contain liquids, such as water, saline, glycerol, and ethanol, as well as substances such as wetting agents, emulsifying agents, or pH buffering agents. Liposomes can also be used as carriers.

A method of eliciting an immune response against a disease or pathogen in a vertebrate (e.g., vaccination) comprises administering an immunogenic composition comprising a multilayer film comprising an RSV epitope. In one embodiment, the polyelectrolyte containing the RSV epitope is in the most exterior or solvent-exposed layer of the multilayer film. The immunogenic composition can be administered orally, intranasally, intravenously, intramuscularly, subcutaneously, intraperitoneally, sublingually, intradermally, pulmonary, or transdermally, either with or without a booster dose. Generally, the compositions are administered in a manner compatible with the dosage formulation, and in such amount as will be prophylactically and/or therapeutically effective. Precise amounts of immunogenic composition to be administered depend on the judgment of the practitioner and may be peculiar to each subject. It will be apparent to those of skill in the art that the therapeutically effective amount of an immunogenic composition will depend, inter alia, upon the administration schedule, the unit dose of antigen administered, whether the compositions are administered in combination with other therapeutic agents, and the immune status and health of the recipient. A therapeutically effective dosage can be determined by the ordinary skilled medical worker based on patient characteristics (age, weight, sex, condition, complications, other diseases, etc.), as is well known in the art. Furthermore, as further routine studies are conducted, more specific information will emerge regarding appropriate dosage levels for treatment of various conditions in various patients, and the ordinary skilled worker, considering the therapeutic context, age and general health of the recipient, is able to ascertain proper dosing.

The immunogenic composition optionally comprises an adjuvant. Adjuvants in general comprise substances that boost the immune response of the host in a non-specific manner. Selection of an adjuvant depends on the subject to be vaccinated. Preferably, a pharmaceutically acceptable adjuvant is used. For example, a vaccine for a human should avoid oil or hydrocarbon emulsion adjuvants, including complete and incomplete Freund's adjuvant. One example of an adjuvant suitable for use with humans is alum (alumina gel). A vaccine for an animal, however, may contain adjuvants not appropriate for use with humans.

It is contemplated that an immune response may be elicited via presentation of any protein or peptide capable of eliciting such a response. In one embodiment, the antigen is a key epitope, which gives rise to a strong immune response to a particular agent of infectious disease, i.e., an immunodominant epitope. If desired, more than one antigen or epitope may be included in the immunogenic composition in order to increase the likelihood of an immune response.

As used herein, “layer” means a thickness increment, e.g., on a template for film formation, following an adsorption step. “Multilayer” means multiple (i.e., two or more) thickness increments. A “polyelectrolyte multilayer film” is a film comprising one or more thickness increments of polyelectrolytes. After deposition, the layers of a multilayer film may not remain as discrete layers. In fact, it is possible that there is significant intermingling of species, particularly at the interfaces of the thickness increments. Intermingling, or absence thereof, can be monitored by analytical techniques such as ζ potential measurements, X-ray photoelectron spectroscopy, and time-of-flight secondary ion mass spectrometry.

“Amino acid” means a building block of a polypeptide. As used herein, “amino acid” includes the 20 common naturally occurring L-amino acids, all other natural amino acids, all non-natural amino acids, and all amino acid mimics, e.g., peptoids.

“Naturally occurring amino acids” means glycine plus the 20 common naturally occurring L-amino acids, that is, alanine, valine, leucine, isoleucine, serine, threonine, cysteine, methionine, aspartic acid, asparagine, glutamic acid, glutamine, arginine, lysine, histidine, phenylalanine, ornithine, tyrosine, tryptophan, and proline.

“Non-natural amino acid” means an amino acid other than any of the 20 common naturally occurring L-amino acids. A non-natural amino acid can have either L- or D-stereochemistry.

“Amino acid sequence” and “sequence” mean a contiguous length of polypeptide chain that is at least two amino acid residues long.

“Residue” means an amino acid in a polymer or oligomer; it is the residue of the amino acid monomer from which the polymer was formed. Polypeptide synthesis involves dehydration, that is, a single water molecule is “lost” on addition of the amino acid to a polypeptide chain.

As used herein “peptide” and “polypeptide” all refer to a series of amino acids connected one to the other by peptide bonds between the alpha-amino and alpha-carboxy groups of adjacent amino acids, and may contain or be free of modifications such as glycosylation, side chain oxidation, or phosphorylation, provided such modifications, or lack thereof, do not destroy immunogenicity. As used herein, the term “peptide” is meant to refer to both a peptide and a polypeptide or protein.

“Substrate” means a solid material with a suitable surface for adsorption of polyelectrolytes from aqueous solution. The surface of a substrate can have essentially any shape, for example, planar, spherical, rod-shaped, etc. A substrate surface can be regular or irregular. A substrate can be a crystal. A substrate can be a bioactive molecule. Substrates range in size from the nanoscale to the macro-scale. Moreover, a substrate optionally comprises several small sub-particles. A substrate can be made of organic material, inorganic material, bioactive material, or a combination thereof. Nonlimiting examples of substrates include silicon wafers; charged colloidal particles, e.g., microparticles of CaCO3 or of melamine formaldehyde; biological cells such as erythrocytes, hepatocytes, bacterial cells, or yeast cells; organic polymer lattices, e.g., polystyrene or styrene copolymer lattices; liposomes; organelles; and viruses. In one embodiment, a substrate is a medical device such as an artificial pacemaker, a cochlear implant, or a stent.

When a substrate is disintegrated or otherwise removed during or after film formation, it is called “a template” (for film formation). Template particles can be dissolved in appropriate solvents or removed by thermal treatment. If, for example, partially cross-linked melamine-formaldehyde template particles are used, the template can be disintegrated by mild chemical methods, e.g., in DMSO, or by a change in pH value. After dissolution of the template particles, hollow multilayer shells remain which are composed of alternating polyelectrolyte layers.

A “capsule” is a polyelectrolyte film in the form of a hollow shell or a coating surrounding a core. The core comprises a variety of different encapsulants, for example, a protein, a drug, or a combination thereof. Capsules with diameters less than about 1 μm are referred to as nanocapsules. Capsules with diameters greater than about 1 μm are referred to as microcapsules.

“Cross linking” means the formation of a covalent bond, or several bonds, or many bonds between two or more molecules.

“Bioactive molecule” means a molecule, macromolecule, or macromolecular assembly having a biological effect. The specific biological effect can be measured in a suitable assay and normalizing per unit weight or per molecule of the bioactive molecule. A bioactive molecule can be encapsulated, retained behind, or encapsulated within a polyelectrolyte film. Nonlimiting examples of a bioactive molecule are a drug, a crystal of a drug, a protein, a functional fragment of a protein, a complex of proteins, a lipoprotein, an oligopeptide, an oligonucleotide, a nucleic acid, a ribosome, an active therapeutic agent, a phospholipid, a polysaccharide, a lipopolysaccharide. As used herein, “bioactive molecule” further encompasses biologically active structures, such as, for example, a functional membrane fragment, a membrane structure, a virus, a pathogen, a cell, an aggregate of cells, and an organelle. Examples of a protein that can be encapsulated or retained behind a polypeptide film are hemoglobin; enzymes, such as for example glucose oxidase, urease, lysozyme and the like; extracellular matrix proteins, for example, fibronectin, laminin, vitronectin and collagen; and an antibody. Examples of a cell that can be encapsulated or retained behind a polyelectrolyte film are a transplanted islet cell, a eukaryotic cell, a bacterial cell, a plant cell, and a yeast cell.

“Biocompatible” means causing no substantial adverse health effect upon oral ingestion, topical application, transdermal application, subcutaneous injection, intramuscular injection, inhalation, implantation, or intravenous injection. For example, biocompatible films include those that do not cause a substantial immune response when in contact with the immune system of, for example, a human being.

“Immune response” means the response of the cellular or humoral immune system to the presence of a substance anywhere in the body. An immune response can be characterized in a number of ways, for example, by an increase in the bloodstream of the number of antibodies that recognize a certain antigen. Antibodies are proteins secreted by B cells, and an immunogen is an entity that elicits an immune response. The human body fights infection and inhibits reinfection by increasing the number of antibodies in the bloodstream and elsewhere.

“Antigen” means a foreign substance that elicits an immune response (e.g., the production of specific antibody molecules) when introduced into the tissues of a susceptible vertebrate organism. An antigen contains one or more epitopes. The antigen may be a pure substance, a mixture of substances (including cells or cell fragments). The term antigen includes a suitable antigenic determinant, auto-antigen, self-antigen, cross-reacting antigen, alloantigen, tolerogen, allergen, hapten, and immunogen, or parts thereof, and combinations thereof, and these terms are used interchangeably. Antigens are generally of high molecular weight and commonly are polypeptides. Antigens that elicit strong immune responses are said to be strongly immunogenic. The site on an antigen to which a complementary antibody may specifically bind is called an epitope or antigenic determinant.

“Antigenic” refers to the ability of a composition to give rise to antibodies specific to the composition or to give rise to a cell-mediated immune response.

As used herein, a “vaccine composition” is a composition which elicits an immune response in a mammal to which it is administered and which protects the immunized organism against subsequent challenge by the immunizing agent or an immunologically cross-reactive agent. Protection can be complete or partial with regard to reduction in symptoms or infection as compared with a non-vaccinated organism. An immunologically cross-reactive agent can be, for example, the whole protein (e.g., glucosyltransferase) from which a subunit peptide has been derived for use as the immunogen. Alternatively, an immunologically cross-reactive agent can be a different protein, which is recognized in whole or in part by antibodies elicited by the immunizing agent.

As used herein, an “immunogenic composition” is intended to encompass a composition that elicits an immune response in an organism to which it is administered and which may or may not protect the immunized mammal against subsequent challenge with the immunizing agent. In one embodiment, an immunogenic composition is a vaccine composition.

The invention is further illustrated by the following non-limiting examples.

### EXAMPLES

### Materials and Methods

Core and Microparticle: The substrate CaCO3 cores were formed in a controlled co-precipitation reaction with the sodium salt of poly-L-glutamic acid (PGA-Na). The solutions of sodium carbonate and calcium chloride (both containing PGA-Na) were pumped through tubing and were rapidly mixed at 1:1 ratio in a flow reaction. PGA-Na provided a more stable particle and also served as the initial layering step on the CaCO3 microparticle.

The precipitated CaCO3 cores were subjected to electrostatic layer-by-layer (LbL) assembly, in which charged polymers with high net positive or net negative charges were assembled on the surface of CaCO3 microparticles. The assembly is driven by the electrostatic attraction between the soluble polymer and the oppositely charged surface. Poly-1-lysine (PLL, positive charge) and poly-1-glutamic acid (PGA, negative charge) homopolymers were alternately layered to assemble a total of 7 layers on the CaCO3 microparticle, with the 7th layer being PGA to yield a net negative surface charge. The 8th layer is the designed peptide containing a C-terminal poly-lysine tail (K20 (SEQ ID NO: 8)or K20Y (SEQ ID NO: 9)) that is net positively charged and will electrostatically layer on the negatively charged surface of the microparticle.

Homopolymers: Both PLL and PGA are sourced from Sigma. They are synthetically made amino acid chains that are either positively charged (PLL) or negatively charged (PGA).

Designed Peptides: Designed peptides were linearly synthesized by solid phase peptide synthesis (SPPS), a process with repeating cycles of alternating N-terminal deprotection and coupling reactions (C-terminus to N-terminus amino acid addition). The SPPS uses N-terminal FMOC protecting groups for coupling and an onium based chemistry with microwave assisted synthesis. The synthesis method for the peptide used in ACT-1216 was modified to utilize carbodiimide based chemistry for higher microwave temperature assisted synthesis that result in faster peptide coupling times. Once the peptides were synthesized, they were subjected to trifluoroacetic acid cleavage to remove any remaining protecting groups on the peptide chain, like FMOC, and the removal of the peptide from the solid support resin on the C-terminus. After cleavage, the peptides were purified through either a C4 or C18 column and lyophilized for storage. The peptides in construct ACT-1190/1211 and ACT-1193/1216 underwent an additional oxidation reaction step to help the peptide fold on the region that contains the four free cysteines. After this oxidation step, the peptides were purified before lyophilization.

ELISA PROTOCOL: ELISA plates were coated with RSV A2 G protein (a generous gift from Ralph Tripp, Univ. of GA) or RSV-G peptide (SEQ ID NO: 3) for 2 hours at room temperature, blocked with ELISA buffer (PB S+1% BSA) for 1 hour, and then washed three times with PBS-T. Mouse serum samples serially diluted in buffer were added and incubated for 2 hours at room temperature or refrigerated overnight. Plates were washed three times with PBS-T and then the HRP-conjugated αmouse IgG secondary antibody was added for 1 hour. Plates were again washed three times with PBS-T. TMB solution was added and color was allowed to develop for 3 minutes before being stopped by the addition of 2M H2SO4. Plates were read immediately at an OD of 450 nm.

ELISPOT PROTOCOL: ELISPOT plates were coated with mouse IL-5 or IFNγ capture antibody overnight at 4° C. Wells were blocked with RPMI Complete +10% FBS at room temperature for 2 hours. Spleens were processed into single cell suspensions in RPMI medium and 5×104 cells/well were added. RSV-M2 (ACT-2019) or RSV-G (ACT-2183) peptides were diluted in RPMI medium and added at final concentrations of 5 and 2.5 μg/ml, respectively. ConA (2 μg/ml final concentration) was added to some wells as a positive control. After an overnight incubation (16-20 hours) at 37° C./5% CO2, the cell suspensions were discarded, and the wells were washed twice with deionized water and then three times with PBS-T. IL-5 or IFNγ biotinylated detection antibody in PBS/1% BSA was added, and the plates were incubated at room temperature for 2 hours. The detection antibody was discarded, and the wells were washed three times with PBS-T. Streptavidin-AP solution was added, and the plates were incubated at room temperature for 45 min to 1 hour, then washed three times with PBS-T and twice with PBS. BCIP/NBT solution was added, and the plates were incubated in the dark for 5-10 min. Plates were rinsed with distilled water to halt color development and then allowed to dry at room temperature. Spots were quantified using the automated Viruspot reader and results were presented as the number of spots per 10₆ spleen cells.

### Example 1: Dose Response of RSV Particles

TABLE 1
SEQUENCES OF THE RSV PARTICULATE COMPOSITIONS

| Construct | DP/Epitope | Sequence | SEQ ID NO: |
|---|---|---|---|
| ACT-1190 | ACT-2182/GM2 | NFVPCSICSNNPTCWAICKRIPNKKPG | 10 |
| | | KKTSGSESYIGSINNITKQSASVASGS | |
| | | K₂₀ | |
| ACT-1193 | ACT-2192/Pam3Cys.GM2 | Pam3CysSKKKKNFVPCSICSNNPTCW | 6 |
| | | AICKRIPNKKPGKKTSGSESYIGSINN | |
| | | ITKQSASVASGSK₂₀ | |
| ACT-1213 | ACT-2129/G | NFVPCSICSNNPTCWAICKRIPNKKPG | 11 |
| | | KKTK₂₀Y | |
| ACT-1214 | ACT-2033/M2 | ESYIGSINNITKQSASVAK₂₀Y | 12 |
| ACT-1211 | ACT-2182/GM2 | NFVPCSICSNNPTCWAICKRIPNKKPG | 13 |
| | | KKTSGSESYIGSINNITKQSASVASGS | |
| | | K₂₀ | |
| ACT-1216 | ACT-2192/Pam3Cys.GM2 | Pam3CysSKKKKNFVPCSICSNNPTCW | 7 |
| | | AICKRIPNKKPGKKTSGSESYIGSINN | |
| | | ITKQSASVASGSK₂₀ | |

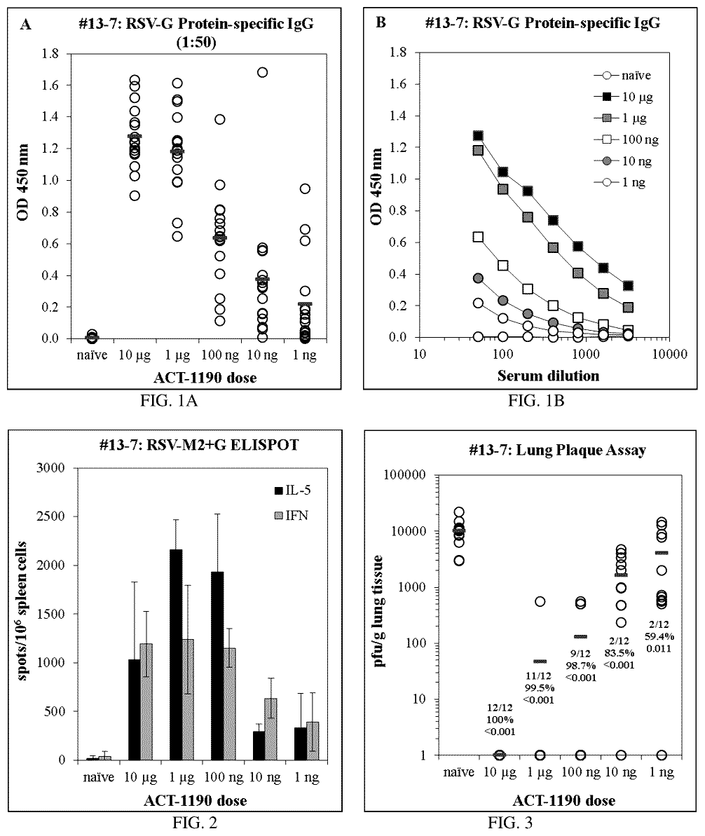

This study examined the potency, immunogenicity, and efficacy of ACT-1190 particles over a dose range of 5 logs. BALB/c mice were immunized on days 0 and 21 with ACT-1190 concentrations ranging from 10 μg to 1 ng of DP. Post-boost sera were examined by RSV-G protein ELISA and post-boost T-cell responses were examined in IL-5 and IFNγ ELISPOT. The results in FIGS. 1A and B show a dose-dependent antibody response across the entire dose range, with only 1-2 outliers in the lower dose groups.

The ELISPOT results are shown in FIG. 2. The IFNγ response increases in a dose-dependent manner from 1 ng to 100 ng and then remains the same up to the 10 μg dose. The IL-5 response peaks at 1 μg.

The remaining mice were challenged with an RSV A2 strain two weeks post-boost. Lungs were harvested 5 days later to measure viral titers by qPCR and plaque assay on Vero cells. The results of the plaque assay are shown in FIG. 3. A significant reduction in viral burden in comparison to naïve mice was achieved at all doses, ranging from 59.4% at the 1 ng dose to complete protection at 10 μg. Although all immunized groups were statistically different from the naïve group, there was no statistical difference between the 10 μg group and either the 1 μg or the 100 ng group (P>0.05 in each comparison). A similar pattern was observed when the samples were analyzed by qPCR (data not shown), although the magnitude of differences is routinely greater in the plaque assay than in the qPCR. The plaque assay is generally more sensitive since it measures actual replicative virus while the qPCR measures total viral M2 gene expression which does not necessarily correlate with viral replication.

### Example 2: PAM3Cys Improves Potency of RSV Particles

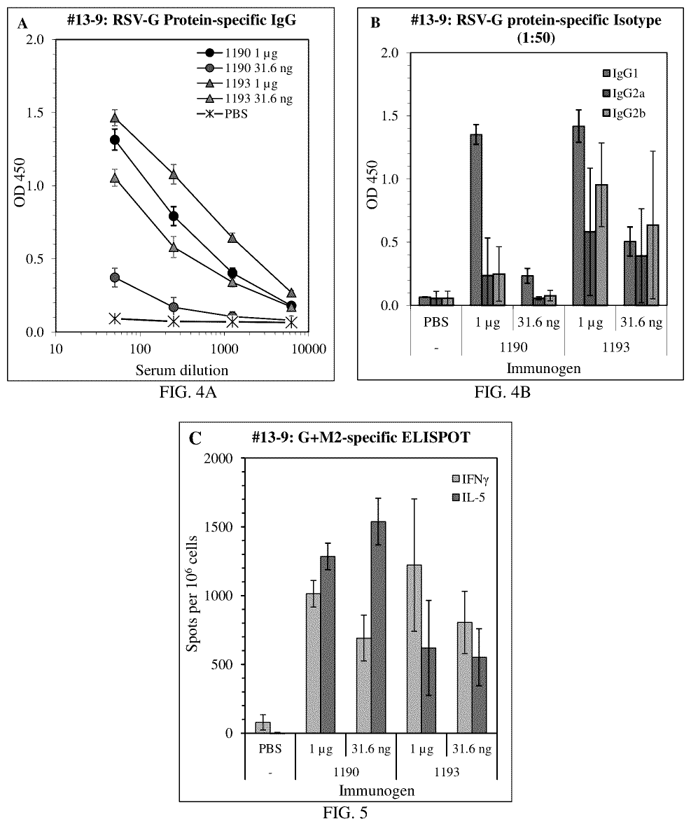

ACT-1193-01 contains Pam3Cys-modified designed peptide (DP) analogous to the unmodified DP in ACT-1190. Groups of BALB/c mice were immunized with 1 μg or 31.6 ng of ACT-1190 (GM2) or ACT-1193 (Pam3Cys.GM2) via f.p. on days 0 and 21. Sera were collected on day 28 for determination of RSV-G protein-specific antibody titers by ELISA. FIG. 4A shows that ACT-1190 and ACT-1193 constructs elicited RSV-G protein-specific antibody responses in a dose-dependent manner, and both constructs elicited predominantly IgG1 (Th2-associated), while ACT-1193 elicited some IgG2a (Th1-associated) and IgG2b isotypes (FIG. 4B). The results show that RSV particles carrying a DP modified with Pam3Cys (ACT-1193) elicit higher antibody titers and a broader isotype distribution than control particle ACT-1190 carrying the unmodified DP.

On day 28, T-cell responses were measured by ELISPOT. Mice immunized with either construct mounted equivalent IFNγ T-cell responses, while the Pam3Cys-modified ACT-1193 elicited fewer IL-5 T-cells than the non-modified ACT-1190 (FIG. 5).

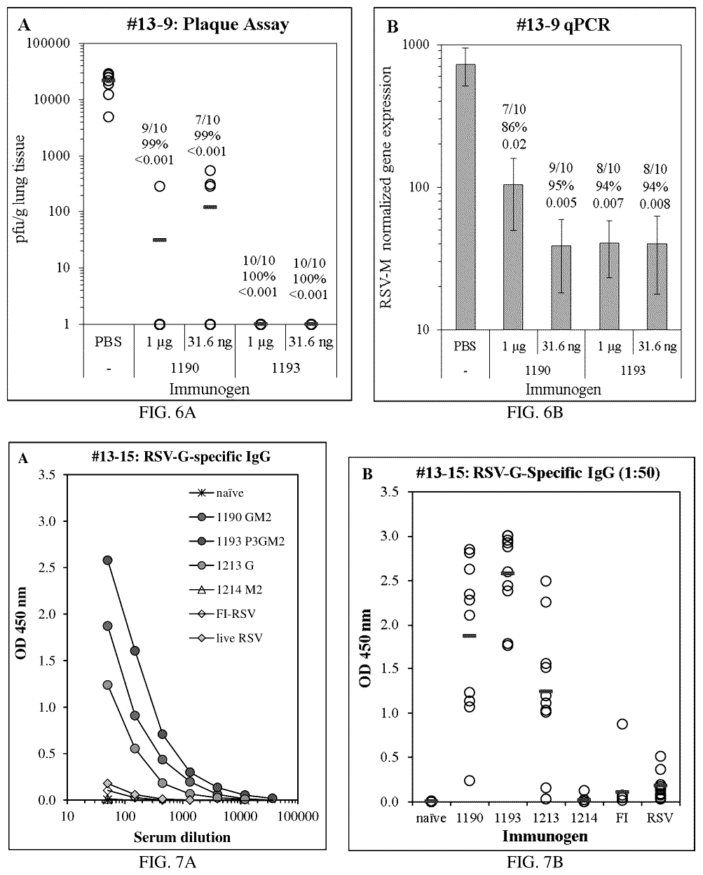

The remaining 10 mice/group were challenged with RSV strain A2 on day 37, and viral burden in the lung was measured by qPCR. The results in FIG. 6A and B show that all immunized groups were equally protected from viral challenge when measured by plaque assay (FIG. 6A) or by qPCR (FIG. 6B). Since qPCR measures total RSV M2 gene expression, and not actual viral replication, this assay may yield false positive values. However, the same trend of protection is seen in the plaque assay which measures actual viral replication. These results show that efficacy is potent and detectable at sub-microgram doses with or without Pam3Cys, although the modification does result in protection of 100% of challenged mice even at the lowest dose of 31.6 ng.

This example shows that including Pam3Cys on the designed peptide improved potency (FIGS. 4A and 6) and phenotype of immune response (favors IgG2a and IgG2b in FIG. 4B and IFNγ over IL-5 in FIG. 5).

### Example 3: RSV Enhanced Disease Study

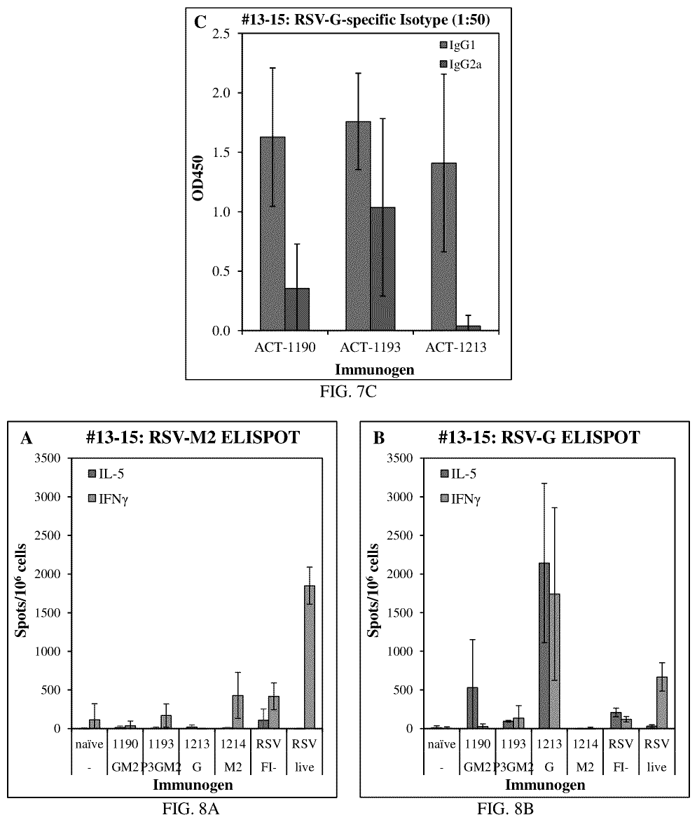

BALB/c mice were immunized with 1 μg of ACT-1190 (GM2), 1 μg of ACT-1193 (Pam3Cys.GM2), 0.67 μg of ACT-1213 (G), or 0.33 μg of ACT-1214 (M2) via f.p. on days 0 and 21. The doses of ACT-1193, ACT-1213 and ACT-1214 were the molar equivalents of 1 μg of ACT-1190. The three control groups included were: FI-RSV (10⁶ pfu equivalent) via i.m. injection on days 0 and 21; 10⁶ pfu of live virus on day 0 (positive); and naïve mice (negative). Post-boost sera were examined by RSV-G peptide ELISA and post-boost T-cell responses were examined in IL-5 and IFNγ ELISPOT. FIGS. 7A and B show that antibody responses in the FI-RSV and live RSV groups were very low in the RSV-G peptide ELISA. The inclusion of M2 (1190) increased the antibody titer (FIG. 7A and B) and induced a shift in the isotype distribution to include IgG2a (FIG. 7C) compared to immunization with the G-only construct (1213), even though the amount of G epitope was the same for both groups. These results confirm that M2 provides both qualitative and quantitative improvement to the antibody response to G. The antibody response was further improved by the Pam3Cys modification of the GM2 DP; note that the ACT-1193 (Pam3Cys.GM2) group has the highest IgG titer (FIG. 7A) and the highest level of IgG2a (FIG. 7C).

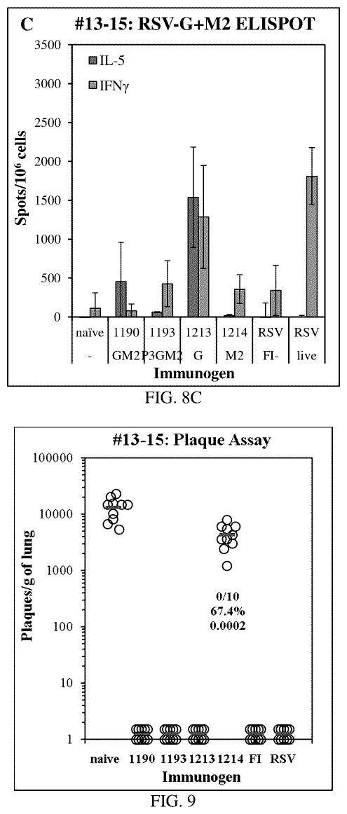

The ELISPOT results are shown in FIG. 8A-C. The IFNγ response to RSV-M2 is dominated by the live RSV group, although positive responses are also seen in the ACT-1193, -1214, and FI-RSV groups. The response to RSV-G is highest in the ACT-1213 group (G only), followed by the ACT-1190 group.

The remaining mice were challenged with RSV strain A2 and lungs from 10 mice per group were harvested 5 days later to measure viral titers by qPCR and plaque assay on Vero cells. The plaque assay results in FIG. 9 show that all mice were completely protected from challenge with the exception of the ACT-1214 group (RSV-M2 alone). The % reduction in viral burden for this group ranged from 41.1% to 90.9%, with an average reduction of 67.4%, as compared to the naïve animals. A similar pattern of protection was observed by qPCR (data not shown).

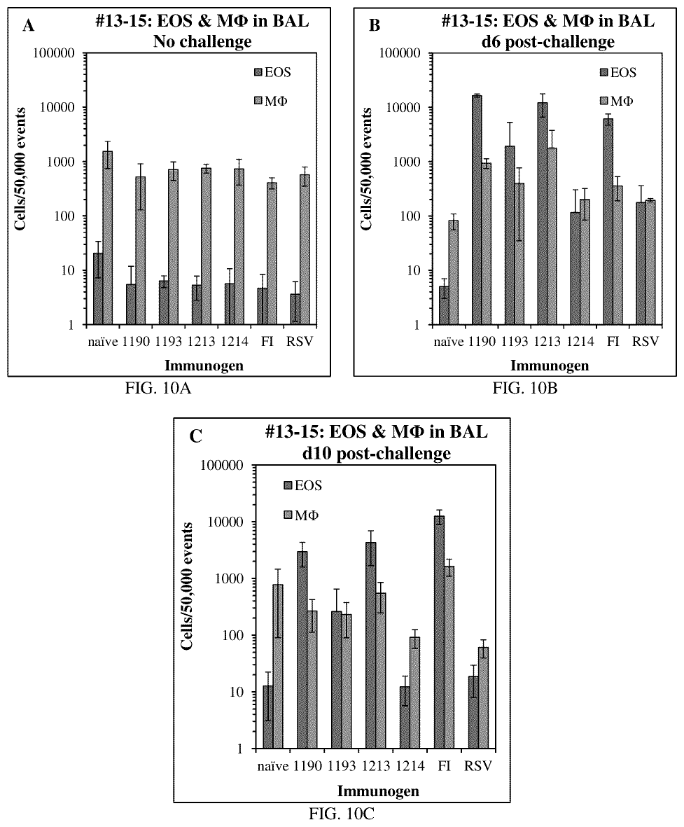

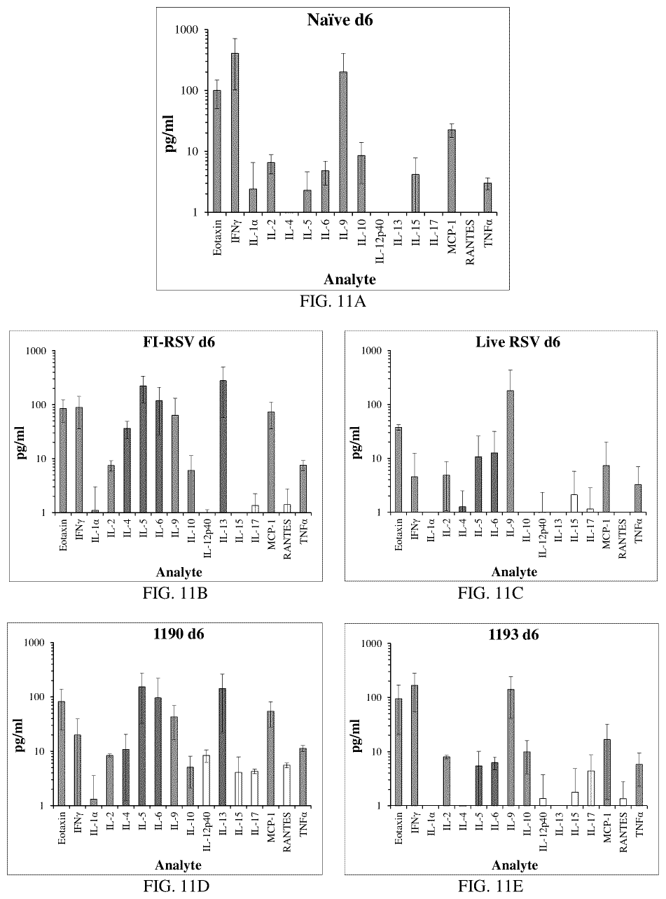

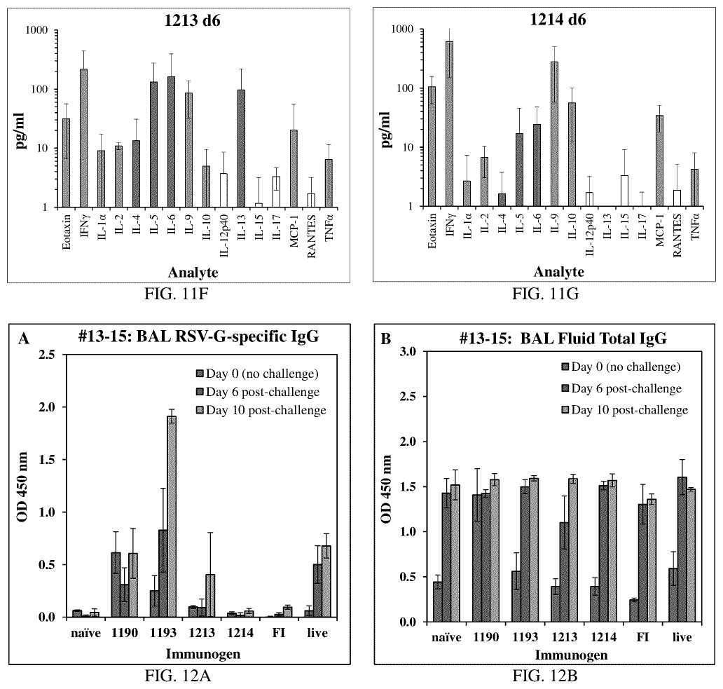

BAL fluid was collected before challenge and then at 6 and 10 days post-challenge (3 mice per group at each timepoint). The BAL cells were stained for CD8⁺/M2-pentamer⁺ cells (at day 6 & 10 post-challenge) and for eosinophils (EOS) and macrophages (MΦ) (at all 3 timepoints). EOS are CD45⁺/SiglecF⁺/CD11c⁻ while MΦ are CD45⁺/SiglecF⁺/CD11c⁺. For each sample, 50,000 events were collected and then a gate was set on CD45⁺ cells, which allowed us to select leukocytes and exclude any red blood cells that escaped lysis. Two-color analysis was then performed on the gated cells. FIG. 10A shows that there is little difference in numbers of EOS and MΦ between groups before the challenge. After challenge, the EOS & MΦ profiles of the ACT-1190 and ACT-1213 groups resemble that of the FI-RSV group (FIGS. 10B and C). The number of EOS in the ACT-1193 group is somewhat elevated in 1 of 3 animals at both post-challenge time points but is overall lower than either ACT-1190 or -1213. The EOS profile of the ACT-1214 group resembles that of the convalescent mice, but the ACT-1214 group is the only group not completely protected from challenge. The results of this study demonstrate that although constructs containing RSV-G DP elicit eosinophil infiltration into the lungs, the constructs provide complete protection from RSV infection in the lung and apparently do not trigger any overt adverse events, and the inclusion of the TLR2 ligand Pam3Cys (ACT-1193) results in modest reduction in eosinophil infiltration in the lungs post-challenge.

### Example 4: RSV Enhanced Disease Study Post-Challenge Lung Cytokine Content

Example 3 confirmed that ACT-1193 (Pam3Cys.GM2) was more potent than ACT-1190 (GM2) and switched the T-cell phenotype from IL-5 to IFNγ, while both constructs provided 100% protection from live RSV challenge. It was particularly interesting to see that immunization with ACT-1190 primed mice for post-challenge lung eosinophil infiltration comparable to that seen in FI-RSV-immunized mice, while immunization with ACT-1193 primed mice for lower levels of post-challenge eosinophil infiltration. 1193-immunized mice also developed higher titer and broader isotype distribution of G-specific antibody responses in the lungs following live RSV challenge. Collectively, these results demonstrate that Pam3Cys-modified LbL-MP elicit immune responses that are quantitatively (antibody titer) and qualitatively (antibody isotype, T-cell phenotype, and eosinophil infiltration post-challenge) improved compared to responses elicited by non-modified LbL-MP. This observation was further strengthened when we examined the cytokine profile of lung (BAL) fluids post-challenge. Cell-free BAL fluids were analyzed for cytokine and chemokine levels by 16-plex Luminex assay. The results from the pre-challenge (day 0) mice were similar to those from the naïve mice on day 6 post-challenge and are not shown for clarity. In all of the day 10 post-challenge groups, most of the cytokines and chemokines were reduced to pre-challenge levels or lower and are also not shown for clarity. The results from all of the day 6 post-challenge groups are shown in FIGS. 11A-G. Columns highlighted in yellow are analytes that are expressed at higher levels in ACT-1190-immunized mice than in FI-RSV-immunized mice following challenge. These include IL-12p40, IL-15, and IL-17 (pro-inflammatory cytokines), and RANTES (chemotactic for leukocytes), suggesting that immunization with RSV-GM2 LbL-MP primes the host for leukocyte infiltration and activation upon subsequent challenge. All of these analytes are also detectable in ACT-1193-immunized mice although at slightly lower levels than in ACT-1190-immunized mice. Columns highlighted in red are Th2-associated cytokines IL-4, IL-5, IL-6, and IL-13, all of which are similarly elevated in the FI-RSV, ACT-1190 (GM2), and ACT-1213 (G) groups, but expressed at lower or undetectable levels in the live RSV, ACT-1193 (Pam3Cys.GM2) and ACT-1214 (M2) groups. Thus, the Th2 cytokine fingerprint in post-challenge BAL fluids differentiates the immunogens into two distinct groups as shown by the vertical alignment of the graphs in FIG. 11A-G. It is particularly interesting to see that IL-4 and IL-13 are elevated in the FI-RSV and ACT-1190 groups but lower or completely absent in the live RSV and ACT-1193 groups. Both of these Th2 cytokines have been associated with eosinophil recruitment in lung inflammatory diseases such as asthma, thus they may be important surrogate markers of RSV-enhanced disease that is usually characterized by Th2-associated inflammation. These results demonstrate that Pam3Cys modification of the GM2 construct favors a Th1 non-inflammatory response that may correlate with lower levels of enhanced disease post-challenge.

### Example 5: RSV Enhanced Disease Study Lung Antibody Responses

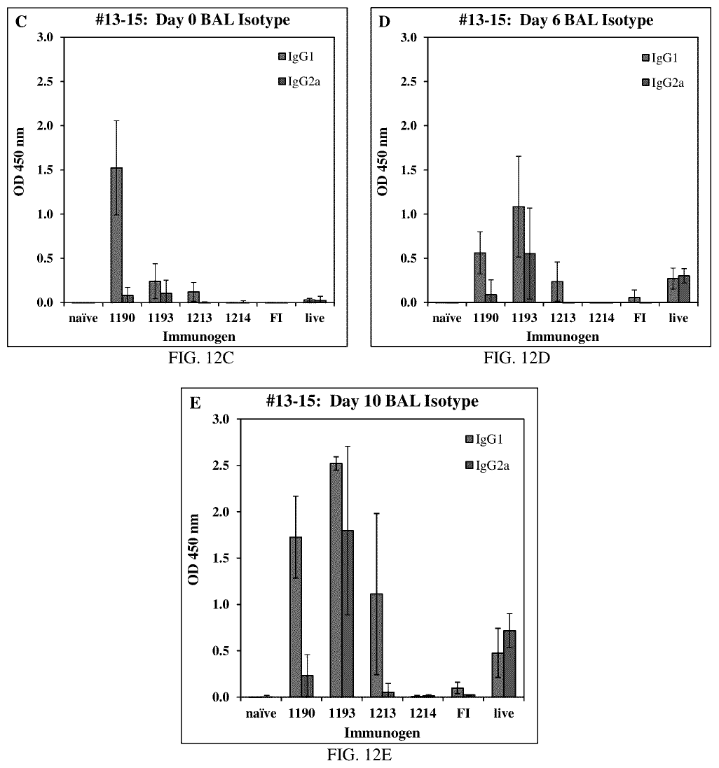

BAL fluid samples were tested for RSV-G peptide-specific IgG and isotypes in ELISA. Total (non-specific) IgG levels were also measured by ELISA for normalization purposes. The BAL was measured at a 1:5 dilution in all ELISAs. FIG. 12A shows that the amount of RSV-G specific antibody increases dramatically over the 10 day post-challenge period in the ACT-1193 group but remains fairly constant in the ACT-1190 group. This pattern is reflected in the isotype ELISA as well. In FIGS. 12C-E, we see that the ACT-1190 group has the highest levels of IgG1 on the day of challenge, but the levels do not increase over time after challenge; the amount of IgG2a is initially low and increases only slightly. In the ACT-1193 group, both IgG1 and IgG2a increase over time. FIG. 12B shows that total IgG levels increase in all groups except the ACT-1190 group in which total IgG was already elevated before challenge. These results agree with previous results showing RSV-G-specific serum IgG2a in the ACT-1193 group but not in the ACT-1190 group.

### Example 6: Second Rsv Enhanced Disease Study Serum Antibody Responses and Efficacy

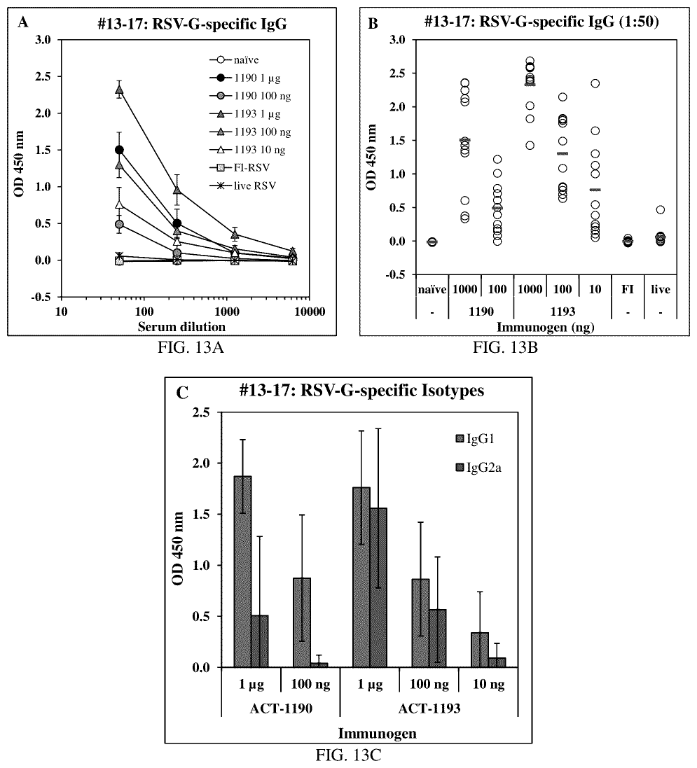

BALB/c mice were immunized with ACT-1190 (GM2) or ACT-1193 (Pam3Cys.GM2) via f.p. on days 0 and 21. The control groups were FI-RSV, live RSV, and naïve mice. The mice were bled 1 week post-boost and RSV-G-specific antibody titers in the sera were measured by ELISA. The results in FIGS. 13A and 13B show that both constructs elicited RSV-G-specific antibody responses in a dose-dependent manner. As previously reported, the addition of Pam3Cys to the DP (ACT-1193) resulted in both higher antibody responses and broader isotype distribution.

Because the mice were bled without the use of anticoagulent, the resulting sera could be tested in an RSV neutralization assay. Sera were pooled and heat-inactivated, serial dilutions were mixed with an equivalent volume of diluted RSV/A2, incubated at 37° C. for 1 hour, and then added to Vero cell monolayers. After 6 days at 37° C., the monolayers were fixed and immunostained. While the anti-RSV-F monoclonal control antibody showed a dose-dependent reduction in the number of viral plaques, only the FI-RSV and live RSV sera showed any neutralization (data not shown). Without being held to theory, it is believed that a higher dose of immunogen needs to be given before neutralizing antibodies can be detected in vitro.

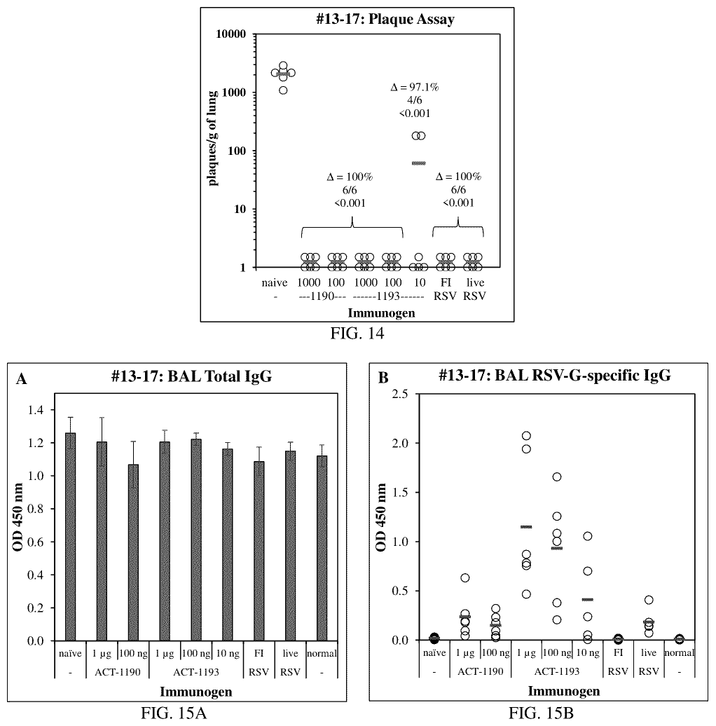

The remaining mice were challenged with RSV/A2 on day 35 and 6 mice per group were sacrificed 5 days post-challenge to assess viral burden in the lungs by plaque assay and qPCR. The remaining 6 mice per group were sacrificed 6 days post-challenge to assess lung histology and BAL cellularity and cytokine content. FIG. 14 shows that the infection levels in the naïve group are fairly consistent, but somewhat lower than desired due to an over-estimation of viral titer of the infecting stock. Nevertheless, protection was 100% in all but the 10 ng ACT-1193 group where infection was detected in 2 of 6 animals. qPCR analysis showed a similar pattern in that only the ACT-1193 10 ng group was not statistically different from the naïve group (data not shown).

### Example 7: Second RSV Enhanced Disease Study Lung Antibody Responses

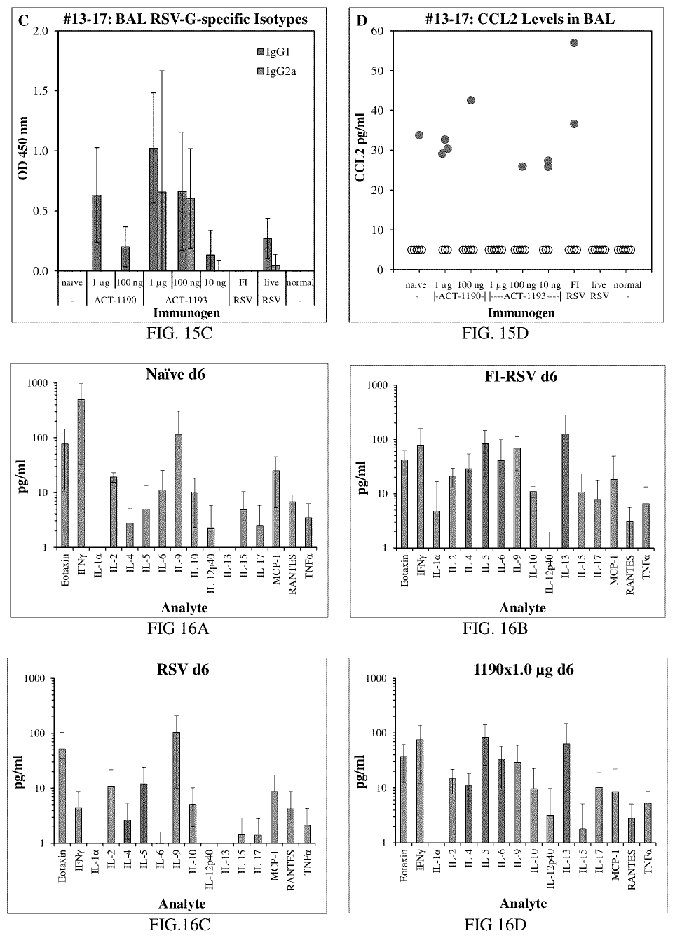

Mice immunized with ACT-1193 had higher serum antibody titers, broader antibody isotype distribution, and lower IL-5 responses than mice immunized with ACT-1190, but equivalent CTL responses and efficacy. There was also a trend toward lower eosinophil counts in the lungs post-challenge. In a second study, BALB/c mice were immunized with ACT-1190 (GM2) or ACT-1193 (Pam3Cys.GM2) via f.p. on days 0 and 21; the control groups were FI-RSV, live RSV, and naïve mice. Mice were challenged with live RSV after the second immunization, and BAL fluid collected from 6 mice per group 6 days after viral challenge was tested for RSV-G peptide-specific antibodies and total IgG by ELISA. We also measured the chemokine CCL2/MCP-1, a Th2-associated chemokine that is known to be involved in lung inflammation, using a sandwich ELISA and following the manufacturer's instructions. Results are shown in FIG. 15. While total IgG levels were roughly equivalent in all BAL samples (FIG. 15A), RSV-G-specific IgG titers were much higher in BAL fluid of mice that had been immunized with ACT-1193 (Pam3Cys.GM2) than those immunized with ACT-1190 (GM2) (FIG. 15B). There was also a shift in RSV-G-specific isotype distribution as BAL from 1193-immunized mice contained both IgG1 and IgG2a, while BAL from 1190-immunized mice contained only IgG1 (FIG. 15C). FIG. 15D shows that CCL2 was detectable in some mice from every group except the group immunized with 1 μg of ACT-1193, the group immunized with live RSV, and the naïve group. The limit of detection in the CCL-2 ELISA is 5 pg/ml; all samples that were above this level are depicted as red circles.

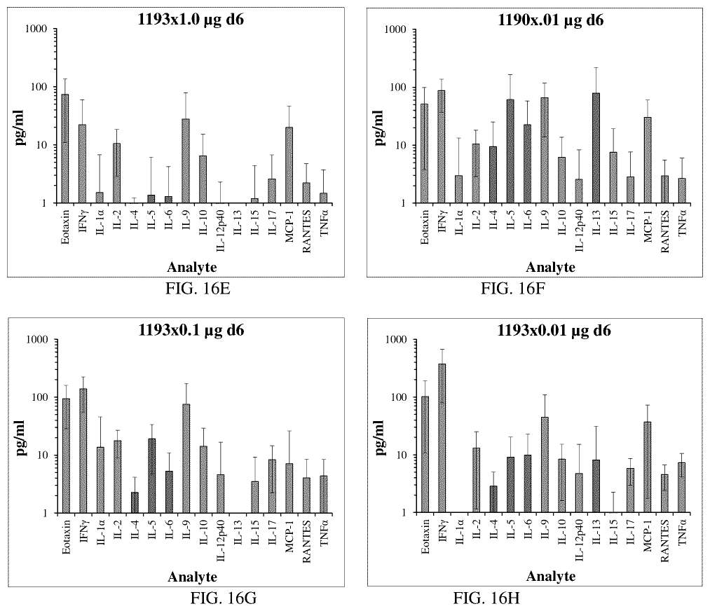

BAL fluids were analyzed by 16-plex ELISA. The results in FIGS. 16A-H are very similar to results obtained with BAL samples from Example 4. Th2 cytokines are highlighted in red. Either dose level of 1190 elicited a Th2 fingerprint identical to that seen in the FI-RSV group. In contrast, the 1193 groups show a dose-dependent decrease in Th2 cytokines, with almost no IL-4, IL-5, IL-6 or IL-13 detected in the 1 μg dose group and increasing amounts in the lower dose groups. The IL-13 response appears to be particularly sensitive to vaccine dose, as this cytokine was not detectable in either the 1 or 0.1 μg 1193 dose group and was lower in the 0.01 μg 1193 group compared to either dose level of 1190.

While the anti-RSV-G IgG response in the sera of 1193-immunized mice is slightly higher than in 1190-immunized mice, the major differences between the two groups are in the serum isotype distribution and the BAL isotype distribution (1193 elicits both IgG1 and IgG2a in both samples), the BAL cytokine profile (1193 inhibits the Th2 cytokine response), the T-cell phenotype (1193 inhibits the IL-5 response [Th2] without affecting the IFNγ response [Th1]), and the BAL CCL-2 response that is present at higher levels in mice immunized with 1 μg ACT-1193 than in mice immunized with 1 μg ACT-1190. This body of evidence clearly demonstrates that LbL-MP loaded with Pam3Cys-modified DP favor a more potent Th1 response than LbL-MP loaded with the same DP minus the Pam3Cys modification. Although CTL activity and efficacy are similar between the two candidates (at doses tested thus far), the reduction in Th2 responses elicited by the Pam3Cys-modified particle may confer protection from post-challenge enhanced disease.

### Example 8: Induction of Chemotaxis-Inhibiting Antibody Responses

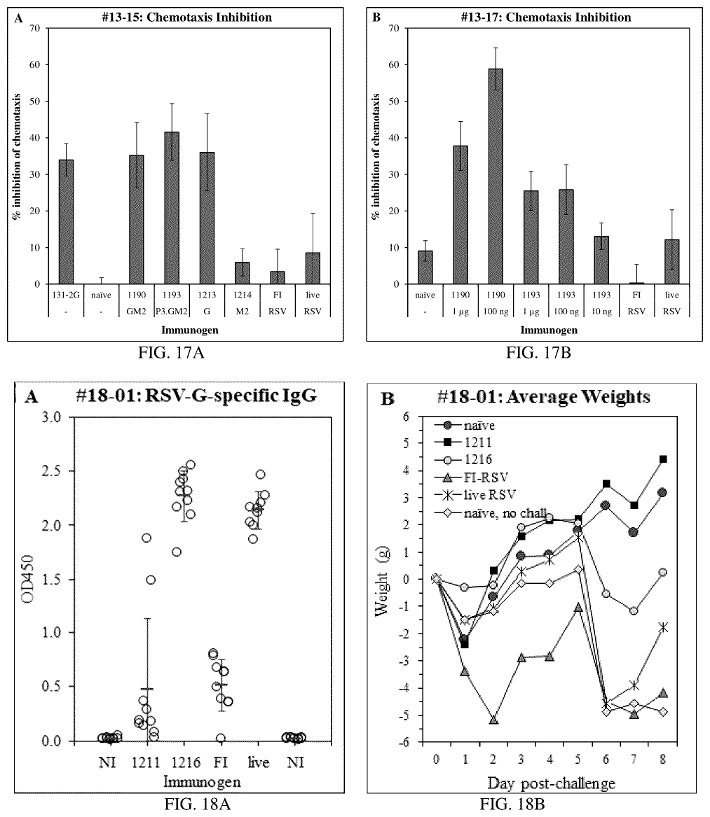

The RSV-G epitope described herein includes a mimic of fractalkine, a CX3C chemokine with chemotactic activity. The particles are designed to elicit antibody responses that not only interfere with viral infection, but also inhibit the inflammatory properties of the RSV-G protein, including chemotaxis or the migration of lymphoid cells toward the site of inflammation. Sera from the enhanced disease studies were analyzed for inhibition of chemotaxis inhibition. Serum antibodies were immunoprecipitated using a pool of IgA and IgG-conjugated Dynal beads and dialyzed against PBS using a molecular weight cutoff of 100,000. Purified, dialyzed antibodies were incubated with native RSV-G protein in the lower chamber of a modified Boyden chamber, at ⅕ the concentration of purified RSV-G protein. A thawed, rested pool of human lymphocytes from 3 adult donors was added to the upper chamber of the Boyden chamber, and the chambers were incubated overnight. The following day (18-24 hours later), the number of viable lymphocytes that had migrated into the lower chamber were scored, and a chemotactic index (CI) was determined. The % inhibition of migration was determined from this CI. Monoclonal antibody 131-2G was included as a positive control for inhibition of migration; it was incubated with RSV-G protein at 50× concentration. The assay was repeated in duplicate wells each night, with a different pool of lymphocytes used on 3 individual evenings; thus, n=6 for each serum sample. The results in FIGS. 17A and B show that antisera raised against any LbL-MP containing the RSV-G epitope inhibited lymphocyte migration by at least 30%, equivalent to the activity in the 131-2G positive control wells. Antisera raised against ACT-1214 (RSV-M2) failed to inhibit chemotaxis, as expected. The inclusion of M2 or the addition of Pam3Cys to the vaccine did not appear to improve the anti-chemotaxis activity above that seen in sera from mice immunized with LbL-MP containing only the RSV-G epitope.

### Example 9: RSV Enhanced Disease Marker Study Antibody Responses and Efficacy

The purpose of this study was to continue the search for reliable markers of enhanced disease. RSV-naïve BALB/c mice (10/group) were immunized i.m. on days 0 and 21 with 1 μg of either ACT-1211 (GM2) or ACT-1216 (Pam3.GM2). FI-RSV and live RSV groups were included as positive and negative controls, respectively. As an added control, a naïve group that would be anesthetized but not challenged was also included. One mouse in the live RSV group expired a few days into the study, reducing the number of mice in this group to 9. Mice were bled 1 week after boost and RSV-G-specific IgG was measured by ELISA against RSV-G peptide (FIG. 18A). The ACT-1211 titers are lower than expected, although this is only the second study testing a 1 μg dose via the i.m. route. Since both the ACT-1211 and ACT-1216 batches used in this study are more than 4 years old, an aliquot of each was recently examined by DLS to determine particle size and dispersity. The results showed that both constructs appear stable with minimal change in size and dispersity (data not shown). Thus, the relatively low antibody responses in the 1211 group may simply be due to study-to-study variability or to the lower dose of 1 μg tested.

Mice were challenged on day 35 and weight loss was monitored for 8 days post-challenge. FIG. 18B shows the group average % change in weight relative to the day of viral challenge (day 0). All groups lost some weight after challenge, including the naïve group that was anesthetized but not given the virus. This indicates that at least a portion of the weight loss can be attributed to the mice having gone under anesthesia. The FI-RSV group did, however, lose the most weight of all the groups, down 5% on day 2, contrasting with previous studies in which the same batch of FI-RSV did not lead to any significant weight loss. A second weight loss event occurred between days 5 and 6. This loss might be attributed to the influx of cells into the lungs that happens at this time. However, this explanation does not account for the weight loss of the naïve animals that were not challenged. Without being held to theory it is believed that this apparent loss could be an artifact, as the average weights from day 6-8 are of the remaining 3/group kept for BAL. In addition, these remaining mice were put into clean cages on day 5 and the loss may simply be due to increased activity in their “new” surroundings.

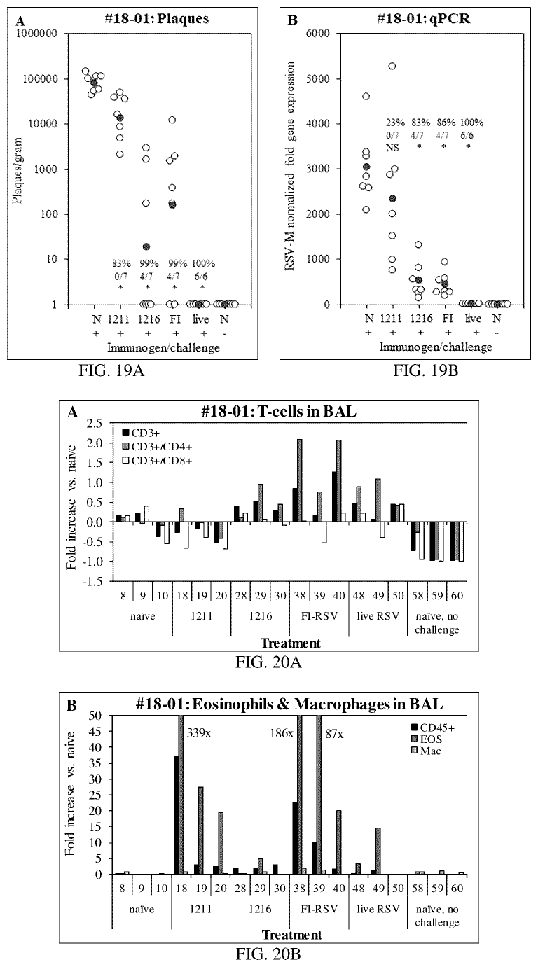

Mice were sacrificed 5 days post-challenge for analysis of lung viral burden and cytokine/chemokine content (6-7 mice/group). Both plaque and qPCR were used to measure viral burden in the lung. The results in FIGS. 19A and 19B show that the efficacy of ACT-1211 is not as high as previously observed, with only a 75.6% average reduction and no individual mice with >90% reduction in viral burden compared to the naïve group. This result correlates with the low serum antibody titers in this treatment group. ACT-1216 and FI-RSV each resulted in significant reduction in viral burden, while prior infection with RSV completely protected the mice from challenge.

The remaining 3 mice/group were sacrificed 8 days post-challenge for analysis of BAL cellularity and cytokine/chemokine content.

### Example 10: RSV Enhanced Disease Marker Study Lung Cellularity and Cytokine Content

Three mice per group were sacrificed 8 days after challenge and BAL fluid was collected for analysis of cytokine and chemokine content and cellularity using markers for T-cells, macrophages, and eosinophils. T-cells were characterized by staining with a cocktail of anti-CD3-FITC/CD4-APC/CD-8-PE, gating on CD3⁺ cells, and measuring CD4⁺ and CD8⁺ cells within the gate. A similar strategy was used to characterize eosinophils (CD45⁺/CD11c⁻/SiglecF⁺) and macrophages (CD45⁺/CD11c⁺/SiglecF⁺). The results are presented in FIG. 20 as fold increase in cell numbers in each group vs. the naïve group. FIG. 20A shows that there is an increase in CD4+ cells in the ACT-1216, FI-RSV, and live RSV groups with the greatest increase in the FI-RSV group. There is an increase in T-cell numbers in all challenged mice relative to the naïve mice that were not challenged. FIG. 20B shows increased numbers of eosinophils in all immunized groups after challenge, to varying degrees. There was a 5-fold increase in eosinophils in one mouse in the ACT-1216 group while 2 mice in the live RSV group showed increases of approximately 3- and 15-fold. The greatest increases in eosinophils by far were in the FI-RSV and ACT-1211 groups. The insets in FIG. 20B show the fold increases for the three values that are above the scale of the graph.

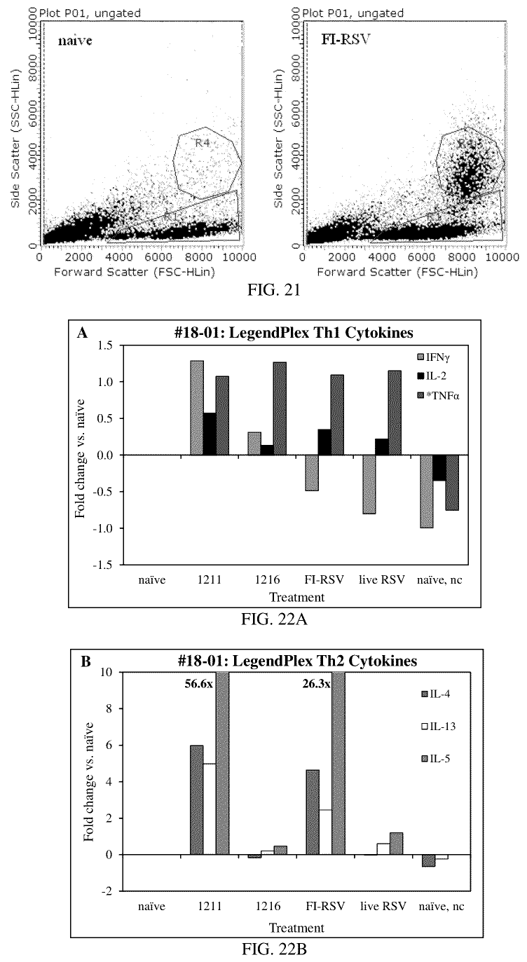

During the analysis of the BAL cells, it was noted that presence of eosinophils could be easily seen in the SSC/FSC dot plots. FIG. 21 shows the scatter plots of one naïve and one FI-RSV mouse. The triangular region (R1) contains the T-cells and was present in all mice in all groups, although with greatly reduced numbers in the naïve mice that were not challenged. The polygonal region (R4) corresponds with larger, more granular cells, i.e., eosinophils and other CD45⁺ cells. The mice that had increased numbers of eosinophils (as determined by the fluorescent markers) had noticeably more cells in R4 of the corresponding scatter plot. Thus, the scatter plots alone give an indication of the presence or absence of infiltrating eosinophils in individual animals.

Th1/2 cytokines and proinflammatory chemokines were measured in both the clarified lung homogenates collected 5 days post-challenge (7/group) and the BAL collected 3 days later (3/group) using multi-analyte flow assay kits from BioLegend. FIG. 22 shows the fold increase or decrease compared to the naïve group for day 5 samples only (most day 8 samples showed little change or a decrease in all analytes compared to naïve controls, and no striking differentiation among treatment groups); asterisks indicate cytokines that are associated with both Th1 and Th2 but are predominantly associated with the phenotype where they are shown. FIG. 22A shows elevated levels of Th1-associated TNFα in all challenged groups, while IFNγ was elevated in only the 1211-immunized group. FIG. 22B shows an increase in Th2-associated IL-4, IL-5, and IL-13 in the 1211 and FI-RSV groups (insets show the fold increases for the IL-5 values that are above the scale of the graph). This pattern is similar to that reported in FIG. 11 and FIG. 13 and again suggests that TLR activation (either TLR2 via Pam3Cys in 1216 or TLR4 via viral proteins in live RSV) dampens the inflammatory cytokine response.

The use of the terms “a” and “an” and “the” and similar referents (especially in the context of the following claims) are to be construed to cover both the singular and the plural, unless otherwise indicated herein or clearly contradicted by context. The terms first, second, etc., as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A composition comprising particles, the particles comprising a multilayer film, the multilayer film comprising two or more layers of polyelectrolytes, wherein adjacent layers comprise oppositely charged polyelectrolytes, wherein one of the polyelectrolytes comprises a designed polypeptide having the structure
(Pam3Cys or Pam2Cys)-(surface adsorption region one)-(RSV-G peptide epitope) -L1-(RSV-M2peptide epitope)-L2-(surface adsorption region two)
wherein surface adsorption region one and two each independently comprise at least two amino acid residues and have the same sign of charge as the designed polypeptide, wherein the net charge per residue of the designed polypeptide is greater than or equal to 0.1,wherein the RSV-M2 peptide epitope includes (ESYIGSINNITKQSACVA) or (ESYIGSINNITKQSASVA), wherein the RSV-G peptide epitope includes (NFVPCSICSNNPTCWAICKRIPNKKPGKKT), and wherein L1 or L2 are linkers comprising 0-100 uncharged amino acid residues;
wherein the polyelectrolytes that are not the designed polypeptide comprise a polycationic material or a polyanionic material having a molecular weight of greater than 1,000 and at least 5 charges per molecule, and
wherein the multilayer film is deposited on a core particle or forms a hollow particle to provide the composition.

2. The composition of claim 1, wherein the composition elicits an IFNγ T-cell phenotype and elicits fewer IL-5-producing T-cells compared to a designed peptide lacking the (Pam3Cys or Pam2Cys).

3. The composition of claim 1, wherein the composition elicits a broader isotype distribution in the RSV-G protein-specific antibody response compared to a designed peptide lacking the (Pam3Cys or Pam2Cys).

4. The composition of claim 3, wherein the composition elicits IgG1, IgG2a and IgG2b isotypes.

5. The composition of claim 1, wherein the designed polypeptide comprises
(SEQ ID NO: 6)
Pam3CSKKKKNFVPCSICSNNPTCWAICKRIPNKKPGKKTSGSESYIGSI
NNITKQSASVASGSKKKKKKKKKKKKKKKKKKKK;
or
(SEQ ID NO: 7)
Pam3CSKKKKNFVPCSICSNNPTCWAICKRIPNKKPGKKTSGSESYIGSI
NNITKQSACVASGSKKKKKKKKKKKKKKKKKKKK

6. The composition of claim 1, wherein L1 and L2 are SGS.

7. The composition of claim 1, wherein two or more of the layers of the multilayer film are covalently cross linked.

8. The composition of claim 7, wherein two or more of the layers of the multilayer film are covalently cross linked by amide bonds.

9. The composition of claim 1, wherein the multilayer film is deposited on a core particle.

10. The composition of claim 1, wherein the multilayer film is in the form of a hollow capsule.

11. A method of administering to an individual in need of immunization from RSV the composition of claim 1.
